(19) United States
(12) Patent Application Publication
(10) Pub. No.: US 2025/0316381 A1
(43) Pub. Date: Oct. 9, 2025

(54) Assessment of Relative Quantitative Effect of Somatic Point Mutations at the Individual Tumor Level for Prioritization

(71) Applicant: Hadasit Medical Research Services and Development Ltd., Jerusalem (IL)

(72) Inventors: Shai Rosenberg, Jerusalem (IL); Jakob (Kobi) Landau, Jerusalem (IL)

(21) Appl. No.: 18/866,860

(22) PCT Filed: Jun. 22, 2023
(86) PCT No.: PCT/IL2023/050651

§ 371 Date: Nov. 18, 2024

Related U.S. Application Data
(60) Provisional application No. 63/354,438, filed on Jun. 22, 2022.

(51) Int. Cl. G16H 50/20; G16B 20/20; G16B 40/20; G16H 50/30

(52) U.S. Cl. CPC G16H 50/20; G16B 20/20; G16B 40/20; G16H 50/30

(57) ABSTRACT

The techniques described herein disclose a method or a system for analyzing genomic data, calculating a predictor and making a quantitative assessment of a biological effect based on the predictor. A biological effect such as the pathogenicity of a cancer a risk that a subject may develop a particular cancer may be determined based on the predictor. The predictor may comprise the observed number of occurrences of a gene variant divided by the expected number of occurrences of the gene variant. The prediction of a drug treatment may comprise prioritization of gene variants according to a selective variant effect and determining which drug treatment to prioritize. The predictions may further comprise using genomic coordinates for each gene variant and nucleotide alterations from various databases, but filtering out duplicate samples from the same subject.

### CROSS-REFERENCE TO RELATED PATENT APPLICATION

The present patent application claims priority to U.S. Provisional Patent Application No. 63/354,438, filed 22 Jun. 2022, and entitled “Assessment of relative quantitative effect of somatic point mutations at the individual tumor level for prioritization”, the disclosure of which is incorporated herein by reference thereto.

### BACKGROUND OF THE INVENTION

Cancer treatment is becoming more precise and personalized to tumors' genomic mutations. Cancer cells are influenced by driver variants with spectral pathogenic effect. These drivers confer selective advantages to the tumors. Currently variants in cancer genes are dichotomized into deleterious or non-deleterious variants. The deleterious variants that can be targeted by biological drugs can be numerous and often not all of them can be targeted to side effects, drug availability and side effects. Currently, no method exists to prioritize which gene/genes should be targeted by drugs.

The identification of many variants in the human genome which could drive disease has been made possible by next generation sequencing technologies. A variety of prediction tools have been proposed to distinguish sequence variants which are causatively neutral from active disease-drivers. Multiple types of data have been promisingly shown to be informative for distinguishing disease-drivers from neutral variants. These, and a variety of other types of data, have been shown to carry information indicating if a variant in the genome could be pathogenic, or neutral in effect, however, evidence has not been produced to show if a particular type of data is actually useful and to what extent.

Therefore, there exists a need for a tool to assist in the identification of new drivers and estimation of mutations' different effects in tumors.

Accordingly, a need arises for techniques that enable better forecasting outcome, therapy selection, and prioritizing of variants more important for the tumor.

### SUMMARY OF THE INVENTION

Aspects of the present disclosure relate to systems and methods for assessing risks of disease (e.g, cancer), predicting treatment response of tumors with specific gene variants and proposing possible forms of treatment based on the assessed risk.

In an embodiment, this disclosure describes a method for quantitatively assessing a biological effect of at least one gene variant of a subject. The method uses a computer system comprising a processor, memory, and instructions stored in the memory, which, when executed by the processor, perform the method comprising a series of steps. The method receives at least one gene variant of the subject. The method analyzes a genomic database to determine a mutation rate for the at least one gene variant. The method determines an observed number of occurrences of the at least one gene variant in the database. The method calculates an expected number of occurrences of the at least one gene variant based on the mutation rate and the observed number of occurrences. The method calculates a predictor associated with the at least one gene variant based on the mutation rate, the observed number of occurrences and the expected number of occurrences. The method uses the predictor to generate a quantitative assessment of a biological effect of the at least one gene variant. Then the computer system transmits the predictor and the quantitative assessment to a user device.

In an embodiment, the quantitative assessment may comprise a prognosis, a risk of developing cancer, or a treatment response. In an embodiment, the predictor comprises a tumor variant amplitude (TVA) equal to a logarithm of a ratio of the observed number of occurrences of the at least one gene variant in the genomic database divided by the expected number of occurrences of the at least one gene variant in the genomic database. In an embodiment, prior to analyzing the genomic database, the genomic database is filtered to avoid duplication of samples from the same subject and also filtered using at least one of: a genomic coordinate of each entry; a nucleotide alteration of each entry; a somatic status of each entry; or a type of cancer of each entry.

In an embodiment, the quantitative assessment may compare a plurality of drug therapies of tumors with gene variants present in the tumors. Based on the comparison, the quantitative assessment may select a drug therapy of the plurality of drug therapies for use with a subject's tumor. In an embodiment, the quantitative assessment may predict, based on the comparison, the likely response of the subject's tumor to the selected drug therapy. In an embodiment, identifying the selected drug therapy of the plurality of drug therapies comprises prioritizing gene variants based on a classification of the gene variants and based on the TVA. In an embodiment, the quantitative assessment may comprise comparing a subject's germline DNA with a database of gene variants and cancer risk and quantifying, based on the comparison, a risk that a subject will develop a cancer. In an embodiment, the quantitative assessment may further comprise comparing a subject's tumor DNA with a database of gene variants and tumor mutations and quantifying a prognosis for a subject. In an embodiment, the method may use the predictor and an artificial intelligence model to determine a diagnosis.

In an embodiment, this disclosure describes a system for quantitatively assessing a biological effect of at least one gene variant of a subject, for use with a user device. The system comprises a measurement device, a processor and memory accessible by the processor and storing computer program instructions which, when executed by the processor, perform a method. The measurement device measures a number of occurrences of the at least one gene variant. The processor analyzes a genomic database to determine a mutation rate for the at least one gene variant. The processor determines an observed number of occurrences of the at least one gene variant in the database. The processor calculates an expected number of occurrences of the at least one gene variant based on the mutation rate and the observed number of occurrences. The processor calculates a predictor associated with the at least one gene variant based on the mutation rate, the observed number of occurrences and the expected number of occurrences. The processor uses the predictor to generate a quantitative assessment of the biological effect of the at least one gene variant. The predictor and the quantitative assessment are transmitted to the user device.

In an embodiment, the quantitative assessment may comprise a prognosis, a risk of developing cancer, or a treatment response. In an embodiment, the predictor comprises a tumor variant amplitude (TVA) equal to a logarithm of a ratio of the observed number of occurrences of the at least one gene variant in the genomic database divided by the expected number of occurrences of the at least one gene variant in the genomic database. In an embodiment, prior to analyzing the genomic database, the processor filters the genomic database to avoid duplication of samples from the same subject and the processor also filters the genomic database using at least one of: a genomic coordinate of each entry; a nucleotide alteration of each entry; a somatic status of each entry; or a type of cancer of each entry.

In an embodiment, the quantitative assessment compares a plurality of drug therapies of tumors with gene variants present in the tumors. Based on the comparison, a drug therapy of the plurality of drug therapies may be selected for use with a subject's tumor. The quantitative assessment may further comprise predicting, based on the comparison, the likely response of the subject's tumor to the selected drug therapy. In an embodiment, identifying the selected drug therapy of the plurality of drug therapies comprises prioritizing gene variants based on a classification of the gene variants and based on the TVA. In an embodiment, the quantitative assessment may compare a subject's germline DNA with a database of gene variants and cancer risk, quantify, based on the comparison, a risk that a subject will develop a cancer and transmit the risk to the user device. In an embodiment, the quantitative assessment may comprise comparing a subject's tumor DNA with a database of gene variants and tumor mutations, and quantifying, based on the comparison, a prognosis for a subject. In an embodiment, the system may use the predictor and an artificial intelligence model to determine a diagnosis.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and the invention may admit to other equally effective embodiments.

FIG. 1 illustrates an exemplary ROC curve for 5,219 variants from MutaGene's benchmark dataset describing the classifiers: MutaGene's occurrences, MutaGene's binomial p-value, the number of occurrences, and the binomial p-value with\without healthy population information inclusion. See also Table 4.

FIG. 2 illustrates a relationship of the total number of different missense drivers (x-axis) and the total number of different nonsense drivers (y-axis) for 535 cancer genes in in the binomial test drivers' catalogue. Each cancer gene is represented as a circle shaded by its role in cancer according to COSMIC; Labels are added to genes with large number of missense or nonsense drivers; TSG represent tumor suppressor genes.

FIG. 3 illustrates the distribution of Clinvar's label amongst 10,866 variants in the extended binomial test drivers' catalogue.

FIG. 4 illustrates the distribution of Cancer Genome Interpreter label amongst 10,866 variants in the binomial test drivers' catalogue. VUS represent variants of unknown significance.

FIG. 5 illustrates Spearman's correlation calculated between 31 computational continuous variant effect predictors and TVA value (raw and imputed) against seven scores from 5 Deep Mutational Scanning (DMS) datasets of TP53 and PTEN genes. Correlations are presented in violin plots and box plots for every DMS score. For Giacomelli's first score (A549_wildtype_Nutilin) correlation only for missense variants in DNA binding domain is shown separately (second from left). TVA and Evolutionary model of Variant Effect (EVE) are labelled on the plot for every DMS score for comparison between TVA and the best score in recent DMS benchmark.

FIG. 6 illustrates Giacomelli's first score of each TP53 variant is plotted against its TVA value. Circles, missense variants; Squares, nonsense variants; Every variant is shaded according to its position in TP53 domains (taken from InterPro); DMS score distribution is presented on the left side; Smooth line with confidence bands are calculated with LOESS method; The Spearman correlation coefficient representing the relationship between the two quantities and its p-value are included in the graph.

FIG. 7 illustrates Kotler's score of each TP53 variant is plotted against its TVA value. Circles, missense variants; Squares, nonsense variants; Every variant is shaded according to its position in TP53 domains (taken from InterPro); DMS score distribution is presented on the left side; Smooth line with confidence bands are calculated with LOESS method; The Spearman correlation coefficient representing the relationship between the two quantities and its p-value are included in the graph.

FIG. 8 is shows Kaplan-Meier curves for OS (overall survival) from diagnosis between TP53 sub-groups as characterized by TVA values and appearance in the catalogue. See Table 6.

FIG. 9 illustrates a forest plot of multivariable Cox regression on TCGA samples with mutated TP53 variant. Age and TVA were analyzed as continuous variables; Two samples were excluded from analysis because they were unique in their cancer type. Cancer type is presented as in TCGA Study Abbreviations.

FIG. 10 illustrates an exemplary drug sensitivity for the PIK3CA gene and PI3K alpha isoform inhibitor (Taselisib). IC50 of cancer cell lines and drug tested from GDSC2 dataset divided by cancer gene sub-groups as characterized by TVA value and appearance in the catalogue. Data is presented in violin plot, box plot and scatter plot for every sub-group. Sub-groups are shaded differently for clearer distinction; Y axis is presented in logarithmic scale; Median values are labelled on the plot for every sub-group; Comparison between sub-groups were done with Wilcoxon signed-rank test.

FIG. 11 illustrates an exemplary drug sensitivity for the PIK3CA gene and PI3K inhibitor (Alpelisib). IC50 of cancer cell lines and drug tested from GDSC2 dataset divided by cancer gene sub-groups as characterized by TVA value and appearance in the catalogue. Data is presented in violin plot, box plot and scatter plot for every sub-group. Sub-groups are shaded differently for clearer distinction; Y axis is presented in logarithmic scale; Median values are labelled on the plot for every sub-group; Comparison between sub-groups were done with Wilcoxon signed-rank test.

FIG. 12 illustrates an exemplary drug sensitivity for the BRAF gene and B-RAF selective inhibitor (PLX4720). IC50 of cancer cell lines and drug tested from GDSC2 dataset divided by cancer gene sub-groups as characterized by TVA value and appearance in the catalogue. Data is presented in violin plot, box plot and scatter plot for every sub-group. Sub-groups are shaded differently for clearer distinction; Y axis is presented in logarithmic scale; Median values are labelled on the plot for every sub-group; Comparison between sub-groups were done with Wilcoxon signed-rank test.

FIG. 13 illustrates an exemplary drug sensitivity for the BRAF gene and the B-RAF selective inhibitor (Dabrafenib). IC50 of cancer cell lines and drug tested from GDSC2 dataset divided by cancer gene sub-groups as characterized by TVA value and appearance in the catalogue. Data is presented in violin plot, box plot and scatter plot for every sub-group. Sub-groups are shaded differently for clearer distinction; Y axis is presented in logarithmic scale; Median values are labelled on the plot for every sub-group; Comparison between sub-groups were done with Wilcoxon signed-rank test.

FIG. 14 illustrates an exemplary drug sensitivity for the PTEN gene and AKT competitive inhibitor (Afuresertib). IC50 of cancer cell lines and drug tested from GDSC2 dataset divided by cancer gene sub-groups as characterized by TVA value and appearance in the catalogue. Data is presented in violin plot, box plot and scatter plot for every sub-group. Sub-groups are shaded differently for clearer distinction; Y axis is presented in logarithmic scale; Median values are labelled on the plot for every sub-group; Comparison between sub-groups were done with Wilcoxon signed-rank test.

FIG. 15 illustrates an exemplary drug sensitivity for the NRAS gene and the MEK1 and MEK2 inhibitor (PD0325901). IC50 of cancer cell lines and drug tested from GDSC2 dataset divided by cancer gene sub-groups as characterized by TVA value and appearance in the catalogue. Data is presented in violin plot, box plot and scatter plot for every sub-group. Sub-groups are shaded differently for clearer distinction; Y axis is presented in logarithmic scale; Median values are labelled on the plot for every sub-group; Comparison between sub-groups were done with Wilcoxon signed-rank test.

FIG. 16 illustrates an exemplary drug sensitivity for the KRAS gene and the BTK inhibitor (Ibrutinib). IC50 of cancer cell lines and drug tested from GDSC2 dataset divided by cancer gene sub-groups as characterized by TVA value and appearance in the catalogue. Data is presented in violin plot, box plot and scatter plot for every sub-group. Sub-groups are shaded differently for clearer distinction; Y axis is presented in logarithmic scale; Median values are labelled on the plot for every sub-group; Comparison between sub-groups were done with Wilcoxon signed-rank test.

FIG. 17 illustrates an exemplary drug sensitivity for the TP53 gene and the MDM2 inhibitor (Nutlin-3a). IC50 of cancer cell lines and drug tested from GDSC2 dataset divided by cancer gene sub-groups as characterized by TVA value and appearance in the catalogue. Data is presented in violin plot, box plot and scatter plot for every sub-group. Sub-groups are shaded differently for clearer distinction; Y axis is presented in logarithmic scale; Median values are labelled on the plot for every sub-group; Comparison between sub-groups were done with Wilcoxon signed-rank test.

FIG. 18 illustrates that total tumor variants count of each TCGA endometrial cancer sample is plotted against its POLE TVA value. Circles, driver variants which appears in the catalogue; Squares, non-driver variants which doesn't appear in the catalogue; Every sample is shaded according to its microsatellite instability (MSI) according to “MSI sensor score”; Large size, POLE related (10a, 10b and 28) single base signatures (SBS) are positive in sample; Small size, POLE related (10a, 10b and 28) single base signatures (SBS) are negative in sample; Smooth line with confidence bands are calculated with loess method; The Spearman correlation coefficient representing the relationship between the two quantities and its p-value are included in the graph.

FIG. 19 illustrates POLE related tumor variants count of each TCGA endometrial cancer sample divided according to POLE sub-groups as characterized by TVA value and appearance in the catalogue. Data is presented in violin plot and box plot for every sub-group. Sub-groups are shaded differently for clearer distinction; Major drivers are labelled on the plot for every sub-group; Comparison between sub-groups were performed using Wilcoxon signed-rank test.

FIG. 20 illustrates an exemplary ROC curve for 4,693 variants from MutaGene's benchmark dataset without BRCA1/2 of MutaGene's occurrences. Curves shown are MutaGene's binomial p-value, the occurrences and the binomial p-value with\without healthy population information inclusion. See also Table 4.

FIG. 21 shows balloon plots representing the residuals of the χ² tests of genes role in cancer categories (according to COSMIC, oncogene and tumor suppressor gene (TSG)) versus type of driver variants (missense/nonsense) in the catalogue. Light shading implies positive correlation between factors, and darker shading implies negative correlation; Circle size is proportional to the amount of the cell contribution.

FIG. 22 shows a Density plot showing the distribution of the catalogue drivers' TVA value for Cancer Genome Interpreter (CGI) known and unknown pathogenic drivers. Comparison between two groups was performed using t test, ˜0=‘****’.

FIG. 23 illustrates the value of Kato's average activity score of each TP53 variant is plotted against its TVA value. High score represents wildtype activity, and low score represents pathogenic activity. Circles, missense variants; Squares, nonsense variants. Every variant is shaded according to its position TP53 domain (taken from InterPro). DMS score distribution is presented on the left side. The Spearman correlation coefficient representing the relationship between the two quantities and its p-value are included in the graph.

FIG. 24 illustrates an exemplary Giacomelli's second score as a function of TVA. Low score represents wildtype activity, and high score represents pathogenic activity. Circles, missense variants; Squares, nonsense variants. Every variant is shaded according to its position TP53 domain (taken from InterPro). DMS score distribution is presented on the left side. The Spearman correlation coefficient representing the relationship between the two quantities and its p-value are included in the graph.

FIG. 25 illustrates an exemplary Giacomelli's third score as a function of TVA. High score represents wildtype activity, and low score represents pathogenic activity. Circles, missense variants; Squares, nonsense variants. Every variant is shaded according to its position TP53 domain (taken from InterPro). DMS score distribution is presented on the left side. The Spearman correlation coefficient representing the relationship between the two quantities and its p-value are included in the graph.

FIG. 26 illustrates the value of Mighell's first score of each PTEN variant is plotted against its TVA value. High score represents wildtype activity, and low score represents pathogenic activity. Circles, missense variants; Squares, nonsense variants. Every variant is shaded according to its position PTEN domain (taken from InterPro). DMS score distribution is presented on the left side. The Spearman correlation coefficient representing the relationship between the two quantities and its p-value are included in the graph.

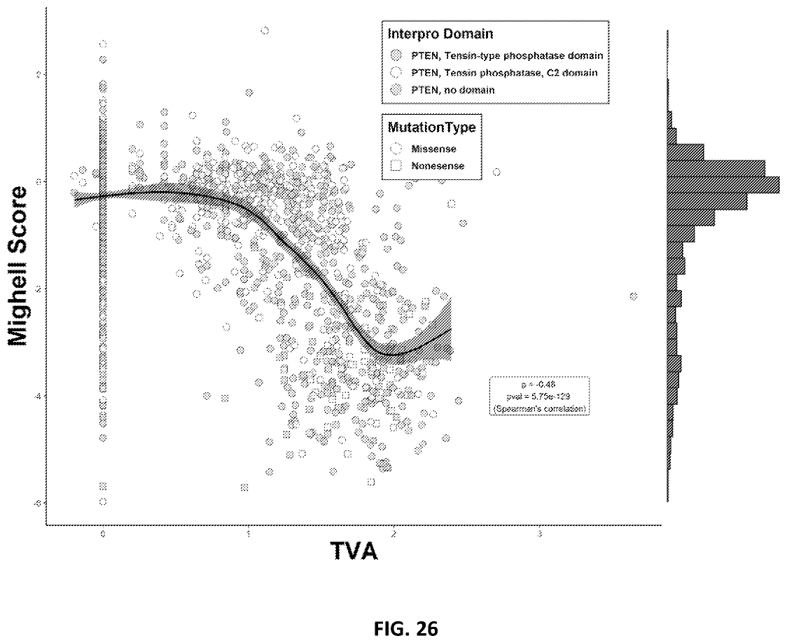

FIG. 27 illustrates an exemplary Matreyek score as a function of TVA. High score represents wildtype activity, and low score represents pathogenic activity. Circles, missense variants; Squares, nonsense variants. Every variant is shaded according to its position PTEN domain (taken from InterPro). DMS score distribution is presented on the left side. The Spearman correlation coefficient representing the relationship between the two quantities and its p-value are included in the graph.

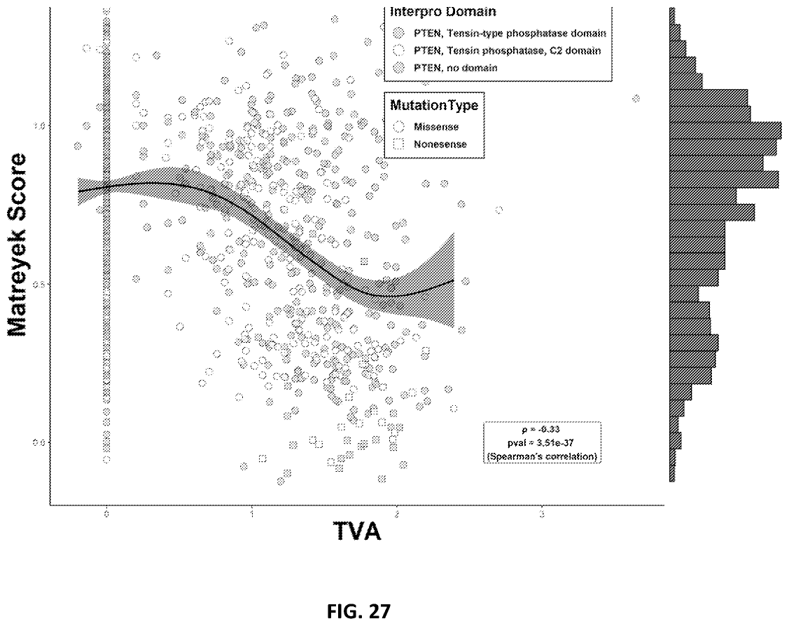

FIG. 28 illustrates the value of Giacomelli's second score (A549_Null_Nut_norm) of each TP53 variant is plotted against its TVA value. Low score represents wildtype activity, and high score represents pathogenic activity. Every variant is shaded according to its context related mutational rate. Large circles represent appearance in the binomial catalogue. The Spearman correlation coefficient representing the relationship between the two quantities and its p-value are included in the graph. Two dashed rectangles highlight two groups with TVA lower than 1.5, (i) pathogenic group with Giacomelli's score above 0.7 and (ii) non-pathogenic with Giacomelli's score between 0.3 to 0.65.

FIG. 29 illustrates the mutational rates of TP53 variants with TVA lower than 1.5 divided according to Giacomelli's second score pathogenic (above 0.7) and non-pathogenic (between 0.3 to 0.65) values. Data is presented in violin and box plots for each group. Groups are shaded differently for clearer distinction; Comparison between groups were performed using t test.

FIG. 30 illustrates an exemplary power analysis estimating the minimal drivers' TVA with power of 0.8 for all trinucleotide-context related mutational rates. Every line represents different mutational rate. The mutational rates range from low mutational rates in lightly shaded lines to high mutational rates in darkly shaded lines. The dashed line represents power of 0.8.

FIG. 31 illustrates Kaplan-Meier curves for overall survival (OS) from diagnosis of Lower Grade Glioma (LGG) TCGA samples between EGFR sub-groups as characterized by TVA value and appearance in the catalogue. See Table 2.

FIG. 32 illustrates TVA's correlation to HRAS clinical subgroups. A distribution of TVA values across HRAS variants subgroups—non-labeled drivers, CS (Costello syndrome), subtle symptoms variants and non-significant non-labeled variants. Violin plot shadings represent the different subgroups. All groups but non-significant also have each variant plotted as a black point. The dot shapes represent—triangle for variant without significance after FDR correction, dot for significance suspected drivers.

FIG. 33 illustrates TVA's correlation to HRAS clinical subgroups as a function of effect of the mutation on the protein. All HRAS labeled variants from HGMD (Human Gene Mutation Database) with y axis ordered by TVA. Point shape represent if the variant is significant in the adjusted binomial test same as in FIG. 32. Point shading represents labels in HGMD. For each point, a label is attached with the amino acid change, with a continuous shading representing amino acid position in HRAS protein.

FIG. 34 illustrates some computer aspects of an exemplary system.

Other features of the present embodiments will be apparent from the Detailed Description that follows.

### DETAILED DESCRIPTION

In the following detailed description of the preferred embodiments, reference is made to the accompanying drawings, which form a part hereof, and within which are shown by way of illustration specific embodiments by which the invention may be practiced. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Unless defined otherwise, all technical and scientific terms used herein have the same meaning as commonly understood by one of ordinary skill in the art to which this disclosure belongs. Although any compositions, methods and materials similar or equivalent to those described herein can be used in the practice or testing of the present invention. All publications mentioned are incorporated herein by reference in their entirety.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### Cancer Variants

The present disclosure relates to methods and systems for estimating which cancer genes will be most useful/effective in predicting optimal treatment and outcomes, including for example reduced tumor size (in response to a drug treatment), remission and the like.

Cancer cells are influenced by driver variants with a spectral pathogenic effect. These drivers confer selective advantages to the tumors. In the treatment of cancer, diagnosis of genetic variants in tumor cells is used for the selection of the most appropriate treatment regime for the individual patient. In breast cancer, for example, genetic variation in estrogen receptor expression or heregulin type 2 (Her2) receptor tyrosine kinase expression determine if anti-estrogenic drugs (tamoxifen) or anti-Her2 antibody (Herceptin) will be incorporated into the treatment plan. In chronic myeloid leukemia (CML) diagnosis of the Philadelphia chromosome genetic translocation fusing the genes encoding the Bcr and Abl receptor tyrosine kinases indicates that Gleevec (STI571), a specific inhibitor of the Bcr-Abl kinase should be used for treatment of the cancer. For CML patients with such a genetic alteration, inhibition of the Bcr-Abl kinase leads to rapid elimination of the tumor cells and remission from leukemia. Furthermore, genetic testing services are now available, providing individuals with information about their disease risk based on the discovery that certain Single Nucleotide Polymorphisms (SNPs) have been associated with risk of many of the common diseases.

In this disclosure, in an example, a Cancer Shared Dataset from several cancer genomic databases may be combined and applied on 535 cancer genes two different measures based on variant's observed and expected frequency based on cancer-specific somatic mutagenesis rates. The first measure is a binary classifier based on a binomial test while the second measure, Tumor Variant Amplitude (TVA), is a continuous measure representing the variants' selective advantage. TVA correlation was examined with many cancer-related experimental and clinical measures. TVA outperformed all other computational tools in its correlation with cancers' mutations experimentally-derived functional scores. It was also highly correlated with drug-response, overall survival, and other clinical implications in relevant cancer genes. This study demonstrates the high impact of a selective advantage measure based on a large cancer dataset, for the understanding of the spectral effect of driver variants in cancer.

### Variant Scoring Techniques

Cancer cells accumulate somatic variants through time. Some variants confer selective advantages, providing cancer cells with improved capabilities such as proliferation, invasion and spreading to other organs, among others. Traditionally, genetic variants in cancer are divided into two distinct categories: driver variants that affect protein activity and contribute to cancer hallmarks, and passenger variants that do not offer advantages to the cancer cells. As this dichotomous classification might be overly simplistic, spectrum-based approaches were proposed to assess the variants' pathogenicity. Such approaches differentiate variants according to quantitative measures such as protein stability and selective pressure. The selective pressure approach defines many variants' subgroups: destructive variants with negative selection, passenger variants with neutral selection, latent driver variants with positive selection in the presence of other same gene driver variants, weak driver variants with moderate positive selection, and strong driver variants with high positive selection. Most pathogenicity scores are accompanied by thresholds providing dichotomous classification due to the simplicity of this approach and the lack of information about variants' quantitative effect. These classifiers' underlying continuous scores are not suitable for the task of forecasting the variants' quantitative effects. Some studies have tried to directly quantify variants' effects through different approaches, but each study has its limitations. One of the best known methods is Envision, a tool based on supervised learning of deep mutational scanning (DMS) datasets. Envision's main limitations are that it is based on small number of good enough DMS experiments and that it mixes information from different experiments and genes with different methods. Another approach is based on evolutional selection intensity. This disclosure's limitations are mainly very small sample size and separation according to cancer types. Part of these quantification tools are superior to classic classifiers in predicting variants' effect(s).

Variant classifiers rely on various features, including protein sequence, evolutionary conservation, structural information, biophysical information, 3D protein clusters, biochemical assays, allele frequency, and tumor variants occurrence. Another method to classify variants is to use genomic context-specific mutational rates. Mutational rates depend on the genomic context and are not constant for specific genomic alterations. Several ways to estimate mutational rates and avoid potential bias may be described. Then, a binomial test can be used to identify tumor variants that are more common than anticipated based on mutational rates. Variants that appear in rates higher than expected are likely to have positive selection in the tumor's evolution process, and thus are more likely to be true drivers of tumorigenesis. Brown et al. (Brown, A. L., Li, M., Goncearenco, A. & Panchenko, A. R. Finding driver mutations in cancer: Elucidating the role of background mutational processes. PLoS Comput. Biol. 15, (2019) (PMID: 31034466) used a binomial test based on trinucleotide context mutational rates to identify new drivers. They reported that this approach showed improved performance compared to the conventional method based on variants occurrences. The main limitations of their study were basing the analysis on a small number of tumor samples, including only samples sequenced against normal tissue, using a small validation dataset, and not comparing their results to healthy population information at all. The binomial test has not yet been used on a large dataset to systematically identify novel drivers.

In this work, the binomial method was implemented on a large, cancer shared dataset (CSD) of 137,224 tumor samples collected from four different sources (TCGA, ICGC, MSKCC and GENIE). Mutational rates, number of sequenced samples, and occurrence of each variant to classify drivers were used to quantify the relative strength or impact of each variant on cancer cells. To quantify this relative strength, a predictor named “Tumor Variant Amplitude” (TVA) was developed which represents the log of the ratio of variants' actual occurrences and the expected occurrences based on mutational rates. TVA was validated as a quantitative predictor of variants' relative strength or impact using experimental, pharmacological, and clinical data. The combination of a binomial test for discovering novel drivers and of TVA for measuring variants' impact on a spectral scale, resulted in a comprehensive and novel catalogue of many somatic drivers. Each driver among 535 selected COSMIC cancer genes, was assigned with a rating of its impact. This catalogue can be useful especially for the long tail of drivers mutated at much lower frequencies compared to mutational hotspots.

In an embodiment, the TVA may be used as part of a system for proposing a treatment based on the prioritized dominant variants of a sample from a patient. The system may access a database of treatments such as medications and may show a healthcare provider a prioritized set of medications based on the variants prioritized by TVA or by another predictor. In an embodiment, artificial intelligence (AI) may employ a predictor as a feature of a set of features for providing a physician with a list of possible diagnoses in relation to a particular patient. In an embodiment, the AI module may comprise a trained model which incorporates information related to the predictor as part of a process of classifying an illness or as part of a process for proposing a treatment of an illness.

### Computer Readable Programming

Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. Thus, it is impossible for any program to damage the execution of any of the other programs running on the system. However, the programs have no way to exchange any information except through the operating system (or by reading files stored on the file system).

Multi-process computing is similar to multi-tasking computing, as the terms task and process are often used interchangeably, although some operating systems make a distinction between the two. The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing.

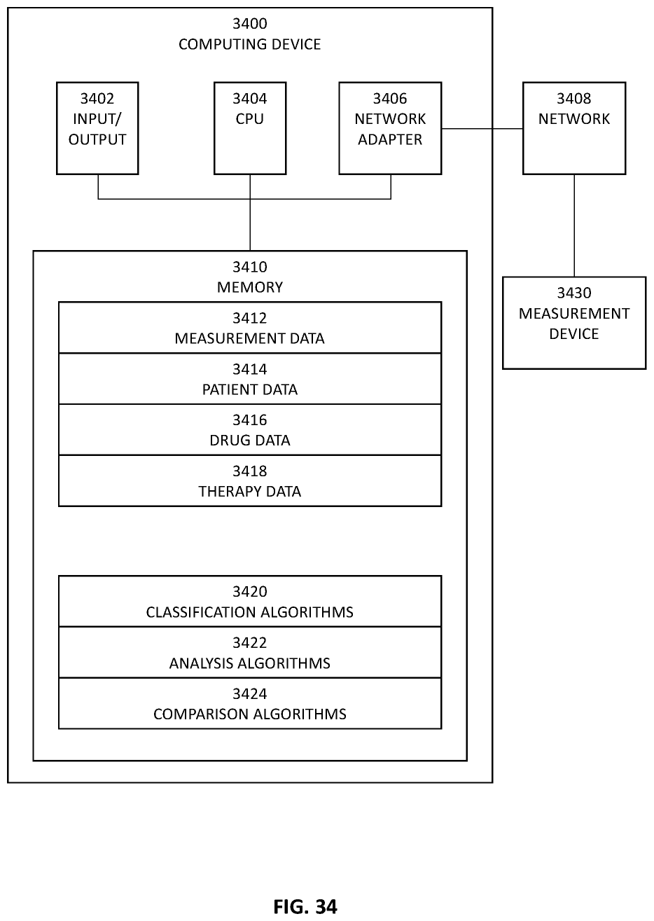

An example of a system is illustrated in FIG. 34. A computing device 3400 is depicted along with a processing unit 3404 (e.g. a central processing unit (CPU), but also encompassing graphics processing units (GPUs) or even multiple processors or cores), an input/output device 3402, a network adapter 3406, and memory 3410. The network adapter 3406 connects the computing device 3400 to a network 3408 which may include a measurement device 3430.

Within the memory 3410 of the computing device 3400 reside data such as measurement data 3412, patient data 3414, drug data 3416, and therapy data 3418. Some data may reside in other locations connected to the network, such as a database of therapeutic treatments or a database of human genes. Also in the memory 3410 of the computing device may reside various programs, sub-routines or algorithms such as classification algorithms 3420, analysis algorithms 3422, and comparison algorithms 3434, amongst others.

A computer readable storage medium, as used herein, is not to be construed as being transitory signals per se, such as radio waves or other freely propagating electromagnetic waves, electromagnetic waves propagating through a waveguide or other transmission media (for example, light pulses passing through a fiber-optic cable), or electrical signals transmitted through a wire. Computer readable program instructions described herein can be downloaded to respective computing/processing devices from a computer readable storage medium or to an external computer or external storage device via a network 3408, for example, the Internet, a local area network, a wide area network and/or a wireless network. The network 3408 may comprise copper transmission cables, optical transmission fibers, wireless transmission, routers, firewalls, switches, gateway computers, and/or edge servers for transmission of data between devices. A network adapter card or network interface 3406 in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks. The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s).

In some alternative implementations, the functions noted in the blocks may occur out of the order noted in the Figures. For example, two blocks shown in succession may, in fact, be executed substantially concurrently, or in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or that carry out combinations of special purpose hardware and computer instructions. Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

From the above description, it can be seen that the present invention provides a system, computer program product, and method for the efficient execution of the described techniques. References in the claims to an element in the singular is not intended to mean “one and only” unless explicitly so stated, but rather “one or more.” All structural and functional equivalents to the elements of the above-described exemplary embodiment that are currently known or later come to be known to those of ordinary skill in the art are intended to be encompassed by the present claims. No claim element herein is to be construed under the provisions of 35 U.S.C. section 112, sixth paragraph, unless the element is expressly recited using the phrase “means for” or “step for.”

While the foregoing written description of the invention enables one of ordinary skill to make and use what is considered presently to be the best mode thereof, those of ordinary skill will understand and appreciate the existence of alternatives, adaptations, variations, combinations, and equivalents of the specific embodiment, method, and examples herein. Those skilled in the art will appreciate that the within disclosures are exemplary only and that various modifications may be made within the scope of the present invention. In addition, while a particular feature of the teachings may have been disclosed with respect to only one of several implementations, such feature may be combined with one or more other features of the other implementations as may be desired and advantageous for any given or particular function. Furthermore, to the extent that the terms “including”, “includes”, “having”, “has”, “with”, or variants thereof are used in either the detailed description and the claims, such terms are intended to be inclusive in a manner similar to the term “comprising.”

Other embodiments of the teachings will be apparent to those skilled in the art from consideration of the specification and practice of the teachings disclosed herein. The invention should therefore not be limited by the described embodiment, method, and examples, but by all embodiments and methods within the scope and spirit of the invention. Accordingly, the present invention is not limited to the specific embodiments as illustrated herein, but is only limited by the following claims.

### EXAMPLES

### Methods

### List of Cancer Genes

The analysis focuses on set of genes from COSMIC cancer census obtained in April 2021. In an example, the work focused on 546 genes that were defined in COSMIC cancer census as having known somatic pathogenic variants and their role is not only as fusion genes. Eleven genes were excluded from the analysis resulting with 535 selected cancer genes. Exclusion of genes was done due to missing information, such as missing transcript and hg19 positions, for these genes (MRTFA, NSD3, NCOA4, MALAT1, TENT5C, NSD2, AFDN, KNL1, SSX2, DEK and NOTCH1). All possible variants for selected genes were obtained from dbNSFP by genes ENSEMBLE coordinates.

### Data Collection

Data was obtained from four different data sources—TCGA, ICGC, GENIE and MSKCC. An API specific for each source was used to download the data (GENIE and MSKCC were downloaded from same database). All variants were converted to hg19 coordinates using the variants' hg19 position and nucleotide alterations from the databases, though other genomic coordinate systems may also be employed. Preprocessing was made to filter out duplicate samples from the same patient, and to check that the somatic validation status and the type of cancer for each variant have been collected.

Variants' specific information for all available variants was collected from dbNSFP v4.2a, a database that compiles many variant predictors scores (sequence based, conservational, variant annotation sources, and meta-predictors) for many possible transcripts (as obtained from VEP, ANNOVAR and snpEff). A summary of allele count and the frequency of each variant in normal populations from gnomAD, ESP6500 and UK10K were also obtained from dbNSFP databases. Preprocessing of dbNSFP was made to separate columns to different transcripts for each gene.

### Drug Response Information Collection

Bulk data of “IC50s Drug Screening” was obtained from Genomics of Drug Sensitivity in Cancer website. Bulk mutation data for cell lines was obtained from Cell Model Passports website.

### TCGA Clinical Data Collection

Clinical data of TCGA samples was obtained from cBioPortal website. Mutational data for all TCGA samples was obtained with cBioPortal API.

### Deep Mutational Scanning (DMS) Experiments Data Collection

PTEN DMS experiments data were obtained from MaveDB, a public repository for datasets from Multiplexed Assays of Variant Effect. TP53 DMS experiments data were obtained from TP53 UMD database.

### Mutational Rate Calculation

For every variant, a trinucleotide context for positive strand was extracted using the Bio.seq module from Biopython v1.75 package. Mutational rates for each of the 96 trinucleotides were defined according to MutaGene mutational rates estimation.

### Transcript Selection

A transcript was chosen for each gene from all possible transcripts according to COSMIC main transcript selection for each gene. If no transcript was selected in COSMIC, the Matched Annotation from NCBI and EMBL-EBI (MANE) transcript was taken from BioMart. Grouping of different nucleotide changes to amino acid changes was performed according to VEP HGVS protein sequence name (HGVSP) in the selected transcript saving only information for the transcript chosen for the gene. For each amino acid change mutational rate was calculated as the sum of all mutational rates of the single base substitution leading to the given amino acid change. CSD Occurrences of all single base substitution leading to the given amino acid change also have been summed.

### Binomial Test Calculation

A one-sided binomial test was performed for every variant, based on the number of samples in CSD=n (samples in CSD which sequenced the variant's gene), variant occurrences in CSD=k (number of samples in CSD with the variant) and mutational rate=p (based on MutaGene's estimated rates). For variants never seen in healthy populations only occurrences of samples were used which were sequenced in comparison to the patient's normal tissue in order to avoid false germline identification. For all other variants occurrences of both samples with and without comparison to normal tissue were used. All calculations were made with SciPy.

### Classifier's Testing

For comparison between MutaGene's estimates and the improved estimates a combined benchmark dataset from MutaGene webserver was used, as also in Brown et al., The dataset from MutaGene's website (https://www.ncbi.nlm.nih.gov/research/mutagene/) was downloaded and various parameters were calculated including: the receiver operating characteristic (ROC) curves, area under the curve (AUC) and maximal Matthew's correlation coefficient (MCC) for: (i) MutaGene's occurrences; (ii) MutaGene's binomial p-value; (iii) the CSD occurrences; (iv) the binomial p-value on all CSD occurrences without any consideration of healthy population information; (v) the binomial p-value with the consideration of healthy population information.

### Deep Mutational Scanning (DMS) Correlations

The Spearman correlation of TVA and cancer genes DMS studies was compared to the correlation of 31 public bioinformatic scores with the DMS scores. Thirty scores were taken from dbNSFP while EVE score was taken from the EVE website (evemodel.org). The scores used are given in Table 3.

### Tumor Variant Amplitude Calculation

For every variant, based on a number of samples in CSD=n (samples in CSD which sequenced the variant's gene), variants occurrences in CSD=k (number of sample in CSD with the variant) and mutational rate=p (based on MutaGene's estimated rates), a statistic named “Tumor Variant Amplitude” (TVA) was calculated using the formula:

$$T V A = log ( \frac{variant actual occurrences}{variant expected occurrences} ) = log ( \frac{k}{n * p} ) .$$

Similar to the binomial test described above, for variants never seen in a healthy population only occurrences of a sample with a comparison to the patient's normal tissue were used. A logarithmic scale was used due to the large tail of distribution of TVA values. The statistic describes the log of the ratio between the actual occurrence of variants and the expected occurrence under neutral selection.
Binomial p-Value Multiple Testing Correction

To correct multiple testing, the False Discovery Rate (FDR) correction was used on all binomial test p-value variants from all selected genes. All calculations were made with statsmodels package.

### Healthy Population Filter

For variants reported in one of the normal genome databases—gnomAD, UK10K or ESP6500—the binomial test and TVA calculation were made based only on occurrences of samples with comparison to normal tissue in order to confirm somatic status and to avoid germline contamination. In addition, variants with combined allele frequency from gnomAD, UK10K and ESP6500 above

$$\frac{1}{{10}^{4}}$$

were filtered out from the drivers' catalogue. This threshold was taken from a former publication in which the prevalence of driver variants in healthy population databases was estimated. (Soussi, T., Leroy, B., Devir, M. & Rosenberg, S. High prevalence of cancer-associated TP53 variants in the gnomAD database: A word of caution concerning the use of variant filtering. Hum. Mutat. 40, 516-524 (2019), PMID: 30720243)

### Passengers Label Definition

In the inspection of drivers' positions in the catalogue, the number of passengers in the same positions (which had drivers) was analyzed. Passengers are defined as variants without significance in the binomial test (p-value>0.1) that also appear in a healthy population database. The statistical significance condition is not enough due to the low power of the binomial test for positions with low mutational rates. Hence, the test might not detect drivers with relatively low TVA values, therefore the healthy population database appearance condition was added as well.

### GDSC's Drug and Genetic Alterations Associations Criteria

The analysis was focused on pairs of drugs and genetic alterations that met the following criteria in GDSC's database: (i) drug response associated with a cancer gene variant (ii) at least 50 cell lines harboring the cancer gene alteration (iii) effect size above 0.7 (above 0.5 indicates a moderate effect size and above 1 indicates a large effect size) (iv) drug-gene association is statistically significant (false discovery rate (FDR) p-value<0.1) and (v) the drug effect on the mutated protein of associated pathways can explain the association.

### Drug Response Subgroups' Definition

For comparison of response to drugs between genes variants' TVA values, samples were binned according to their gene variant TVA score and according to appearance in the drivers' binomial catalogue. Non-drivers were defined as variants absent from the binomial catalogue with TVA≤1.5. This larger TVA value for variants not in the catalog enlarges the number of variants in the non-driver group. Weak drivers were defined as variants in the binomial catalogue with 1≤TVA<2. Moderate drivers were defined as variants in the catalogue with 2≤TVA<3. Strong drivers were defined as variants in the catalogue with 3≤TVA<4. Very strong drivers were defined as variants in the catalogue with TVA≥4. The last group was needed for KRAS, NRAS and PIK3CA genes.

### MSI Threshold

In uterine cancer, TCGA samples with POLE variants were defined as sample positive for microsatellite instability (MSI) according to MSI sensor score. A cutoff of 3.5 was used, as suggested in the MSI score's original paper.

### POLE Drivers' Subgroups' Definition

For comparison between POLE variants' TVA, samples were binned according to their POLE variants with the highest TVA score and appearance in the drivers' binomial catalogue. Non-drivers were defined as POLE variants absent from the binomial catalogue with TVA<=1.5. Weak drivers were defined as POLE variants in the binomial catalogue with 1<=TVA<2. Moderate drivers were defined as POLE variants in the catalogue with 2<=TVA<3. Strong drivers were defined as POLE variants in the catalogue with 3<=TVA<4.

### Survival Analysis Subgroups' Definition and Calculations

In the overall survival analysis, all TCGA samples with more than one unique TP53 variant were excluded. For overall survival comparison between patients with TP53 variants' TVA values samples were binned according to their TP53 TVA score and appearance in the drivers' binomial catalogue. Non-drivers were defined as TP53 variants absent from the binomial catalogue with TVA<=1.5. Weak drivers were defined as TP53 variants in the binomial catalogue with 1<=TVA<2. Moderate drivers were defined as TP53 variants in the catalogue with 2<=TVA<3. Strong drivers were defined as TP53 variants in the catalogue with 3<=TVA<4. Analysis was performed in R with survival package and visualization with survminer package.

### Multivariable Overall Survival (OS) Analysis

A multivariable analysis was carried out of TVA continuous value, age of diagnosis, sex, and cancer type on all TCGA pan cancer samples with unique TP53 variant. Five samples were filtered out due to small sample size in their cancer types: Testicular Germ Cell Tumors (TGCT), Pheochromocytoma and Paraganglioma (PCPG), Diffuse Large B-cell Lymphoma (DLBC). Analysis was performed in R with survival package and visualization with forestmodel package.

TABLE 1
Saturation Mutagenesis Studies design and limitations

| | | | protein | | | |
|---|---|---|---|---|---|---|
| | | | property | | Protein | |
| Study | gene | Platform | of interest | Type of variants | domains | Type of Drivers |
| Kato | TP53 | yeasts | Downstream | Only missense | All domains | — |
| et al. | | | transcription | | | |
| | | | activity | | | |
| Kotler | TP53 | Human cell | Cell Growth | Includes both | Only variants | — |
| et al. | | cultures | | missense and | in the DNA | |
| | | | | nonsense | binding | |
| | | | | | domain | |
| Giacomelli | TP53 | Human cell | Cell Growth | Includes both | All domains | — |
| et al. | | cultures | | missense and | but some | |
| | | | | nonsense but | methods not | |
| | | | | some methods | suited for | |
| | | | | only suited | all domains. | |
| | | | | for missense | | |
| | | | | variants | | |
| Mighell | PTEN | yeasts | Transcription | — | All domains | — |
| et al. | | | activity | | | |
| Matreyek | PTEN | Human cell | Protein | — | All domains | Suited only for |
| et al. | | cultures | abundance | | | thermodynamically |
| | | | (VAMP-seq) | | | unstable variants |

PMID: 12826609, 30224644, 29979965, 29706350, 29785012

TABLE 2
Pairwise comparison of overall survival of
EGFR mutated gliomas using log rank test

| | EGFR | EGFR | EGFR Weak |
|---|---|---|---|
| | wildtype | Passengers | Drivers |
| EGFR Passengers | 0.324 | — | — |
| EGFR Weak Drivers | <2e−16 **** | 0.046 * | — |
| EGFR Moderate Drivers | 7.4e−13 **** | 0.087 + | 0.198 |

~0 = ‘****’,
<0.001 = ‘***’,
<0.01 = ‘**’,
<0.05 = ‘*’,
<0.1 = ‘+’

TABLE 3
Variant Prediction scores list used
in cancer DMS correlation comparison

| Variant Prediction score | Type | Data source |
|---|---|---|
| SIFT | Empirical | dbNSFP |
| bStatistic | Empirical | dbNSFP |
| GERP++_NR | Empirical | dbNSFP |
| GERP++_RS | Empirical | dbNSFP |
| LRT | Empirical | dbNSFP |
| phyloP100way_vertebrate | Empirical | dbNSFP |
| phyloP17way_primate | Empirical | dbNSFP |
| phyloP30way_mammalian | Empirical | dbNSFP |
| SIFT4G | Empirical | dbNSFP |
| SiPhy_29way_logOdds | Empirical | dbNSFP |
| DEOGEN2 | Supervised | dbNSFP |
| FATHMM | Supervised | dbNSFP |
| Fathmm.MKL | Supervised | dbNSFP |
| Fathmm.XF | Supervised | dbNSFP |
| MPC | Supervised | dbNSFP |
| PrimateAI | Supervised | dbNSFP |
| VEST4 | Supervised | dbNSFP |
| fitCons | Unsupervised | dbNSFP |
| GenoCanyon | Unsupervised | dbNSFP |
| phastCons100way_vertebrate | Unsupervised | dbNSFP |
| phastCons17way_primate | Unsupervised | dbNSFP |
| phastCons30way_mammalian | Unsupervised | dbNSFP |
| PROVEAN | Unsupervised | dbNSFP |
| EVE | Unsupervised | https://evemodel.org/ |
| Eigen | Metapredictor | dbNSFP |
| Eigen.PC | Metapredictor | dbNSFP |
| M-CAP | Metapredictor | dbNSFP |
| MetaLR | Metapredictor | dbNSFP |
| MetaSVM | Metapredictor | dbNSFP |
| MVP | Metapredictor | dbNSFP |
| REVEL | Metapredictor | dbNSFP |

### Results

### Example 1: Binomial Test Improvements

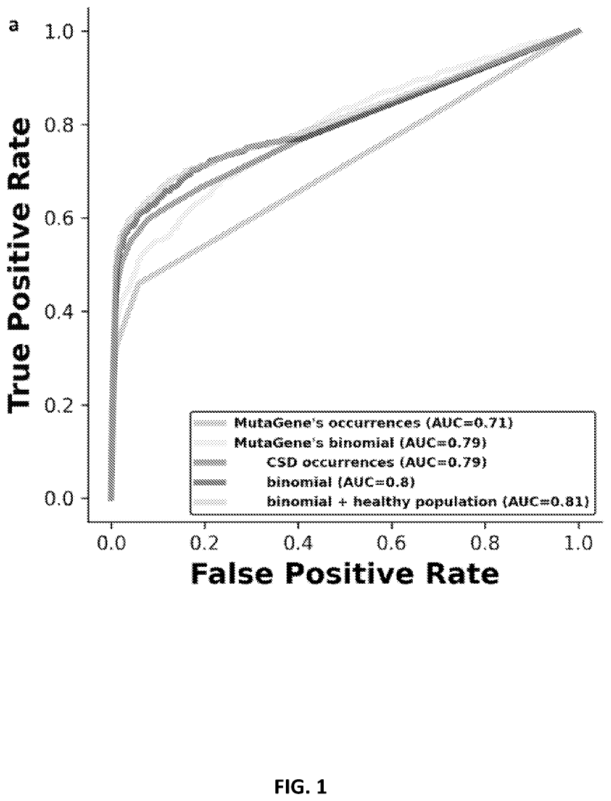

An improved application of the binomial test on cancer gene variants was developed to identify pathogenic variants with positive selection. The major improvements include (i) using healthy population data, thus providing more precise predictions than analysis based solely on occurrences in cancer datasets, (ii) analysis that enables inclusion of samples that were not sequenced against normal tissue as a comparison, thus significantly enlarging the sample size, and (iii) grouping of nucleotide changes that lead to the same amino acid changes, thus focusing on proteins' impact rather than genomic changes. The parameters used for the analysis were variant occurrences in cancer datasets, the number of samples in the cancer datasets, and the estimated mutation rates for each variant's genomic context. Using four different public databases, a 137,224 samples cancer shared dataset (CSD) was created that is about six times larger than previously investigated. Mutation rates were based on MutaGene's pan-cancer context dependent mutational rates estimation. The binomial tests were performed in two different manners: (i) for all variants, all CSD's sample occurrences were included, (ii) including CSD's samples occurrence but for variants appearing in healthy genome database, only CSD's samples occurrences with normal tissue comparison were included. In addition, for the second manner, variants were excluded with allele frequency above 0.0001 in a healthy genome database because they can represent normal genomic variation (see Materials and Methods). This approach was tested against a combined benchmark dataset from the MutaGene webserver used in Brown et al. This combined dataset was derived using five different datasets of experimental assays and contains a total of 5,277 labeled variants from 58 cancer genes. The CSD occurrences approach outperformed both MutaGene's occurrences and MutaGene's binomial p-value in all examined indices (AUC-ROC: 0.7904>0.7083/0.7903) (Table 4, FIG. 1). The binomial p-value without the consideration of a healthy population improved the prediction accuracy compared with the CSD occurrences (AUC-ROC: 0.8025>0.7904) (Table 4, FIG. 1). The binomial p-value with the consideration of healthy population improved the prediction even more (AUC-ROC: 0.8102>0.8025) (Table 4, FIG. 1).

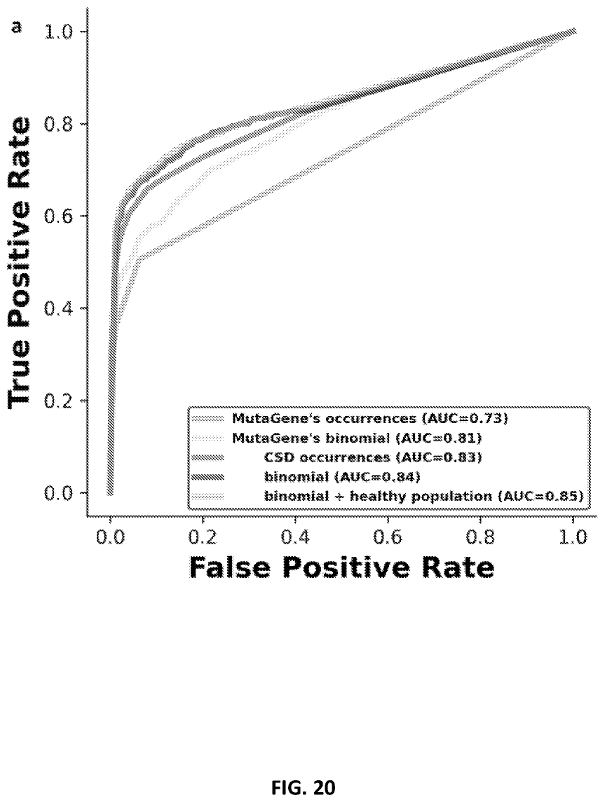

The combined benchmark dataset also includes germline variants, especially from BRCA1 and BRCA2 genes. Some cancer genes such as BRCA1 and BRCA2 are called cancer predisposition genes. These genes are richer in germ line variants compared to somatic variants in cancer. The binomial approach is suited for somatic variants due to its reliance on somatic mutagenesis rate estimates. This makes variants from germline cancer genes less accurate for evaluating the binomial test method. Indeed, when BRCA1 and BRCA2 variants were filtered out from the combined benchmark datasets, the method performed even better (Table 4, FIG. 20).

TABLE 4
Performance comparison on MutaGene's combined benchmark dataset

| | | | | Combined benchmark | | |
|---|---|---|---|---|---|---|
| | Combined benchmark | | | dataset - without | | |
| | dataset - all variants | | | BRCA1/2 | | |
| | AUC- | Max | TPR at | AUC- | Max | TPR at |
| Measure | ROC | MCC | 10% FPR | ROC | MCC | 10% FPR |
| MutaGene's occurrences | 0.7083 | 0.4809 | ~0.5 | 0.7307 | 0.509 | ~0.5 |
| MutaGene's binomial p-value | 0.7903 | 0.5344 | 0.551 | 0.8055 | 0.5661 | 0.58 |
| The CSD occurrences | 0.7904 | 0.5957 | ~0.6 | 0.8286 | 0.6372 | ~0.65 |
| Our binomial p-value (without | 0.8025 | 0.6306 | 0.639 | 0.841 | 0.6752 | 0.696 |
| healthy population) | | | | | | |
| Our binomial p-value (with | 0.8102 | 0.6487 | 0.655 | 0.8464 | 0.688 | 0.709 |
| healthy population) | | | | | | |

AUC-ROC = Area Under the Curve of Receiver Characteristic Operator; MCC = Matthews Correlation Coefficient; TPR = True Positive Rate; FPR = False Positive Rate.

### Example 2: Amino Acid Change Drivers' Catalogue Characteristics

The improved optimal approach with integration of healthy population information was applied on 535 selected cancer genes (see Methods). This approach identified 10,866 suspected amino acid change variants as pathogenic with FDR adjusted p-value threshold of 0.1.

Some tools, such as a first binomial tool and structural cluster tools, predict the pathogenicity of variants according to amino acid position within a gene and mark all different variants in these positions as pathogenic variants. However, gene positioning is not sufficient to define the pathogenic state of variants as some amino acid variants still retain properties of the reference amino acid. All amino acid change variants in the drivers' catalogue were summarized according to gene positions to map how many of the suspected drivers are in a position with other drivers as well. This analysis shows that most variants (71%, n=7,669) are unique drivers in their gene's position and, in one fifth of these positions, there are also passenger variants (Table 5). Passengers were defined as variants that appear in a healthy population at least once and appear in tumors as expected under the null binomial distribution assumption (see Methods). In the other 29% of variants there may be at least one additional driver per position. In this group the higher number of drivers per position is associated with fewer passengers found in these positions, implying that these positions are highly important and less susceptible to changes (Table 5). For each position, the number of passengers was calculated out of all possible non-drivers amino acid changes. The analysis showed that this association is not due to a lower number of possible amino acid changes left after the exclusion of drivers in their positions (Table 5).

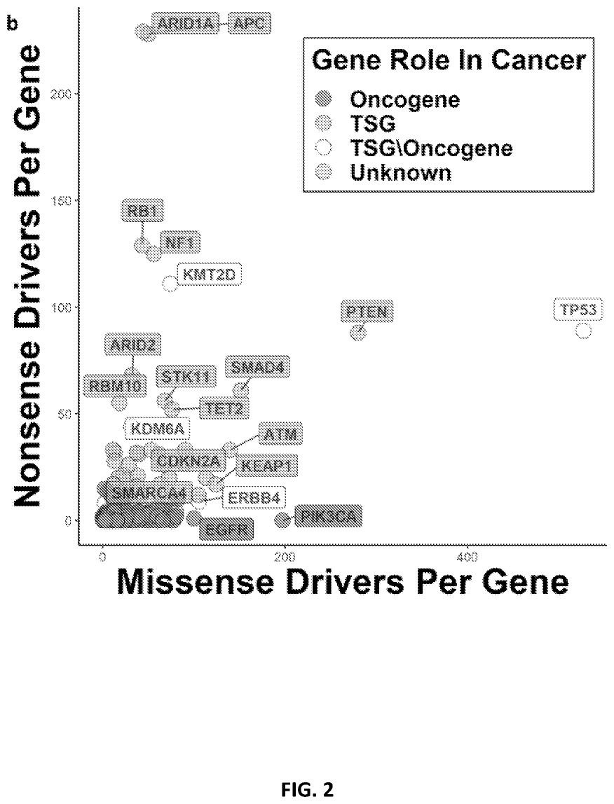

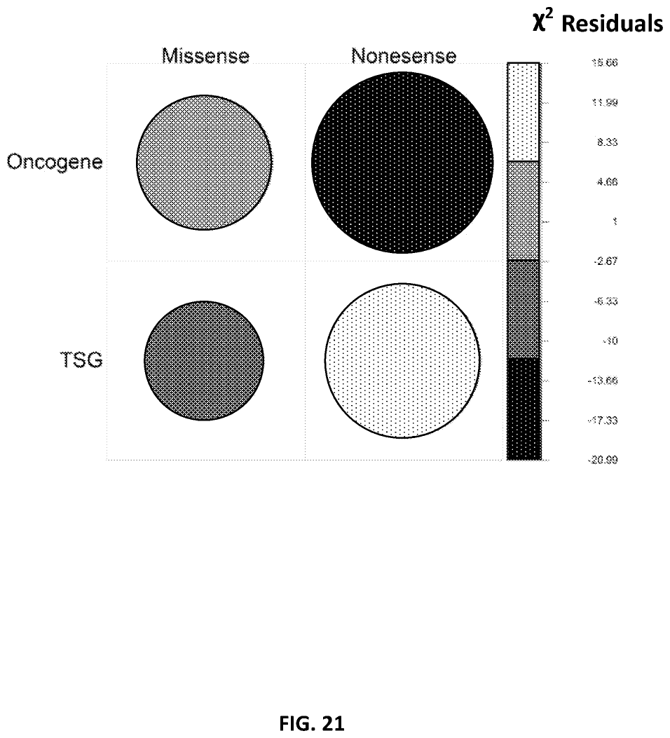

The overall view of drivers' number and type for each gene shows that tumor suppressor genes (TSGs) had larger counts of drivers compared to smaller counts in Oncogenes (OGs) (FIG. 2). Regarding the type of variants, TSGs have both missense and nonsense drivers while Oncogenes have mainly missense drivers and in rare cases a few nonsense drivers as well, (p-value<2.2e-16, Pearson's Chi-squared test) (FIG. 2, FIG. 21).

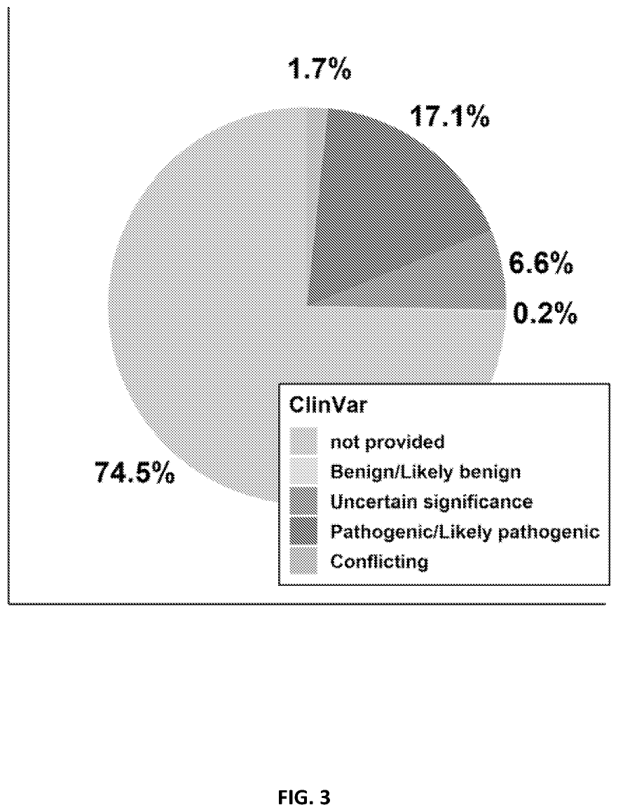

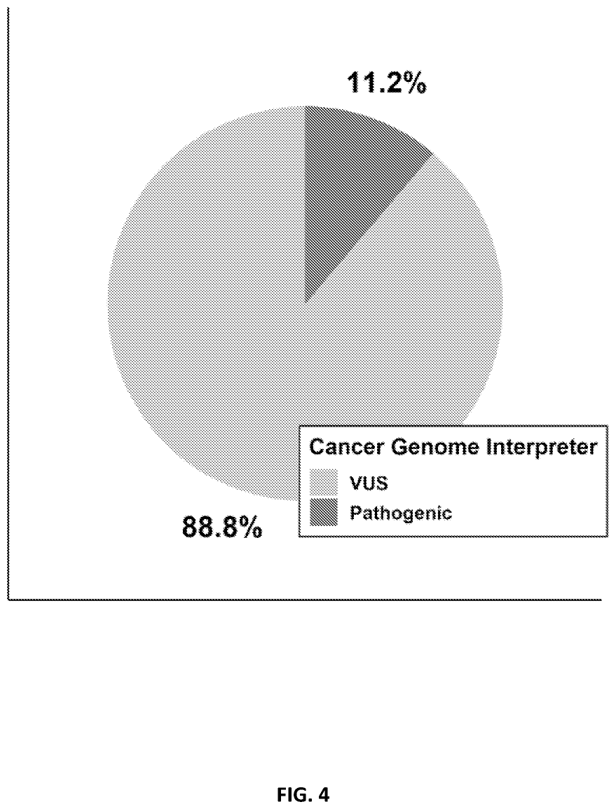

The catalogue of variants was examined in relation to publicly available clinical annotation databases. In ClinVar, which is not specific to cancer, about three quarters of variants identified by the approach are absent; 17% were categorized as “Pathogenic\Likely Pathogenic”; 6.6% were categorized as “uncertain or conflicting” while only 0.2% (n=24) were categorized as “Benign\Likely Benign” (FIG. 3). Most of these “Benign” variants (80%) were submitted by a single submitter, which might suggest less established clinical labeling in ClinVar. This examination confirms the high specificity of the catalogue. In Cancer Genome Interpreter, a cancer specific source of only pathogenic variants consisted of three public sources (ClinVar, OncoKB and DoCM), 88.8% of variants identified by the approach are not reported, and 11.2% categorized as pathogenic in cancer (FIG. 4). This examination emphasizes the ability of the approach to identify many new cancer-related somatic pathogenic variants.

TABLE 5
the catalogue drivers and passengers per gene amino acid positions

| | | Position with any | |
|---|---|---|---|
| Number of | Positions in | passenger (% of | Passengers count out of all |
| Drivers in | catalogue | Positions in | non-drivers possible amino |
| position | (n) | catalogue) | acid changes (%) |
| 1 | 7,790 | 21.9% | 4.9% out of 40,600 |
| 2 | 654 | 8.2% | 2.2% out of 2,674**** |
| 3 | 199 | 7.0% | 2.2% out of 629**** |
| 4 | 110 | 2.7% | 1.3% out of 237** |
| 5 | 69 | 0.0% | 0% out of 88* |
| 6 | 48 | 0.0% | 0% out of 25 |
| 7 | 14 | 0.0% | No non-drivers possible |
| | | | amino acid changes |

Right column—one sided binomial test p-value calculation to compare with the passenger counts of positions with 1 driver (probability 4.9% - first row):
~0 = ‘****’,
<0.001 = ‘***’,
<0.01 = ‘**’,
<0.05 = ‘*’,

### Example 3: Drivers TVA Correlation to Experimental Studies

The question was examined whether there is a quantitative relation between variants' excessive prevalence and their functional activity. P-value is best used to measure statistical significance rather than quantitative measurements. Hence, a metric or statistic was defined that should measure the selective advantage of variants on cancer cells, using the same parameters as in the binomial test. This statistic is called “Tumor Variant Amplitude” (TVA), and it is equal to log

$$( \frac{variant actual occurrences}{variant expected occurrences} ) .$$

It measures the number of tumors in which the variant is observed compared to the number of occurrences which would be expected under no selective pressure. A higher positive TVA value indicates that a variant has a greater selective advantage compared to variants with lower TVA. For variants never seen in the CSD, TVA value is not a finite number, therefore two alternative forms of TVA were examined: (i) raw TVA includes only variants seen at least once in the CSD (ii) imputed TVA in which the TVA value for variants absent from the CSD is defined as 0, representing neutral selection.

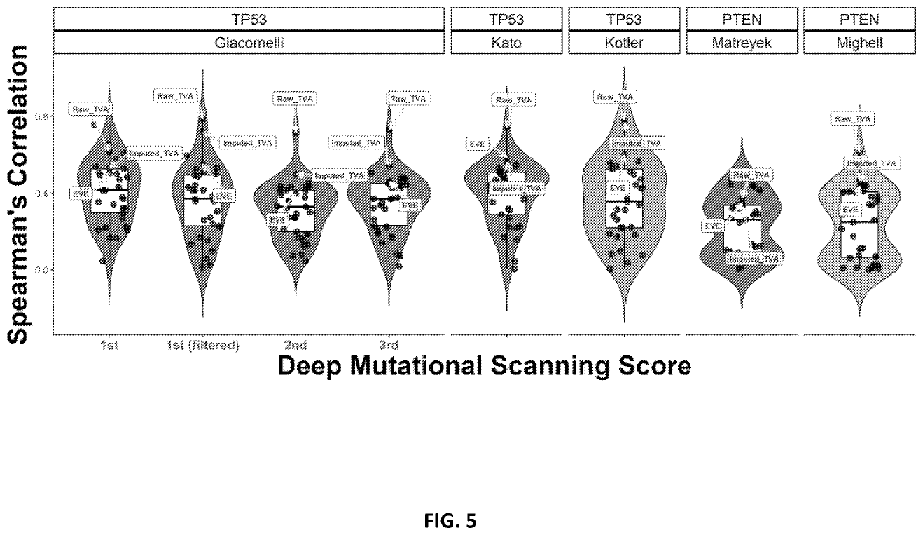

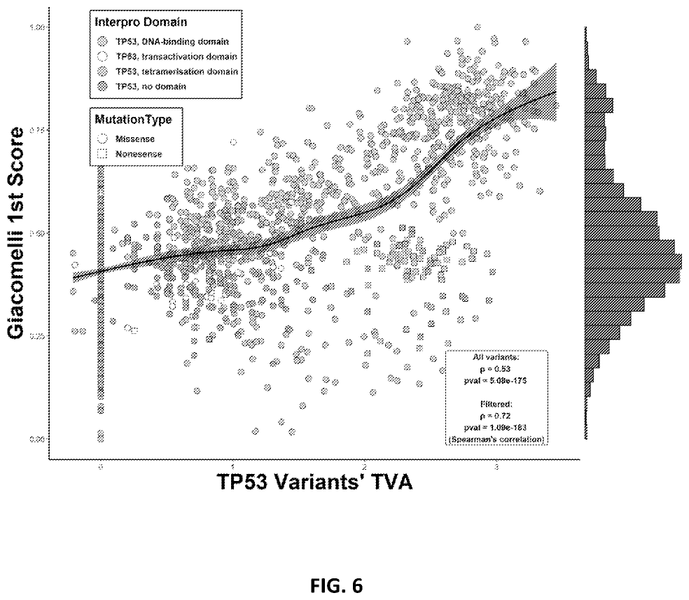

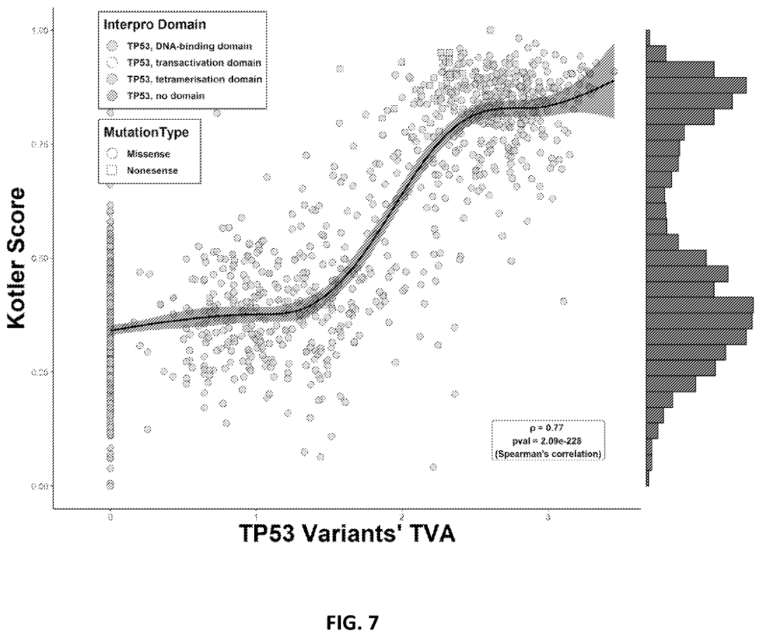

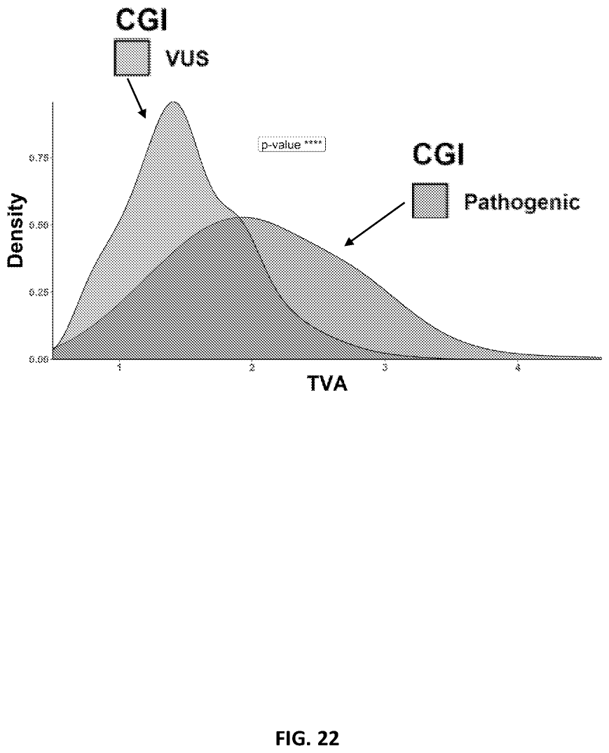

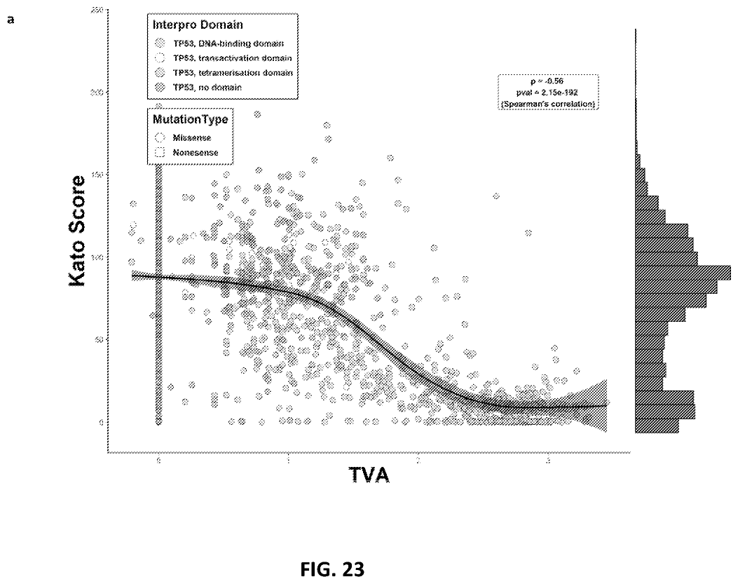

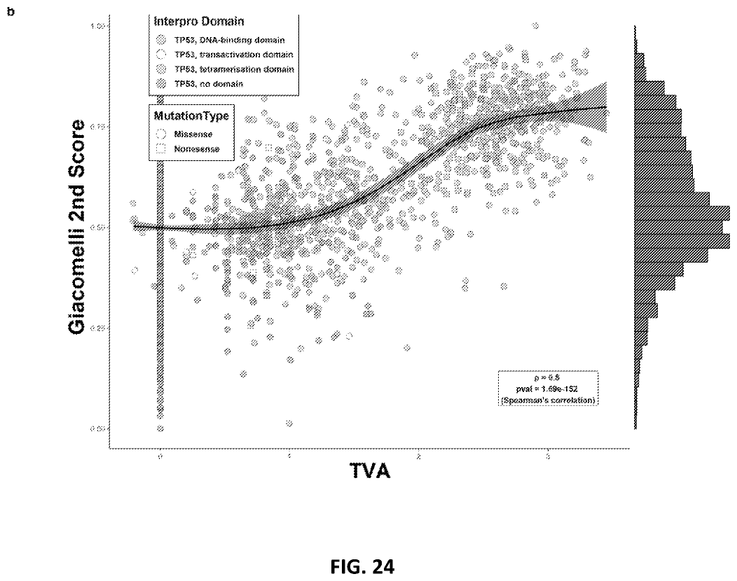

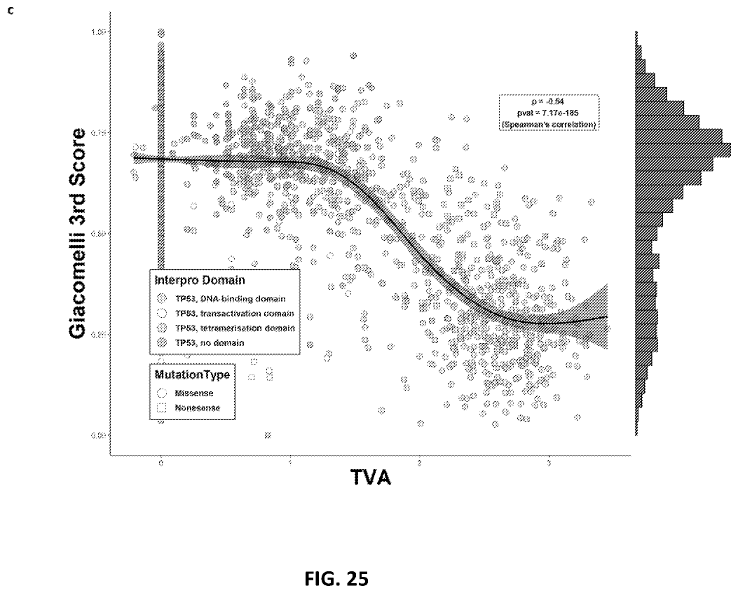

Deep mutational scanning (DMS) experiments are a useful source to quantify variants' effects. A recent study benchmarked many variant effect predictors by statistical correlations to DMS experiments. Data were collected from five DMS studies conducted on cancer genes with many known somatic pathogenic variants and which included a large library of variants in the study. TP53 was the subject of three of the studies and PTEN was the subject of the other two studies. Each of these studies differs in the experimental platform used, the protein property of interest, the type of alterations included, and the protein domain focus. All these differences result in specific limitations in every study (Table 1). Spearman's correlation was calculated between raw TVA and between imputed TVA with the DMS experiments scores. For PTEN in the Mighell study, only variants with high confidence were used. One TP53 study had three scores representing three different experimental measurements, therefore each score was used separately. For comparison Spearman's correlations was also calculated for 30 variant predictors from dbNSFP and a recently published Evolutionary model of Variant Effect (EVE) score (see Materials and Methods). The EVE score is an improvement of the DeepSequence tool that was ranked first in statistic correlations to DMS experiments in a recent comparison of many variant effect predictors. The analysis of all these studies shows a moderate to strong correlation of imputed TVA and cancer related DMS experiments scores (ρ=0.33-0.77, Spearman's correlation), and an even stronger correlation for raw TVA in all DMS studies (ρ=0.38-0.79, Spearman's correlation) (FIG. 5). In the comparison to 31 predictors, imputed TVA was ranked first in four out of the seven DMS scores examined, while for the remaining three scores it ranked second, seventh and ninth (FIG. 5). In the Kato study imputed TVA was ranked second after the EVE score but raw TVA was much higher than the EVE score. It should be noted that one of the PTEN studies where TVA was ranked ninth is considered less accurate since it measured protein stability which is not the same as functional activity. TP53 Giacomelli's first score in which TVA was ranked seventh is one of three assays from the same paper that is known as inadequately screening for nonsense variants and variants located outside of the DNA binding domain (FIG. 6). It seems that the differences of performance of TVA among the three Giacomelli scores occur because the first score is based on cancer cells with wildtype TP53 compared to the other two scores which are based on cancer cells with null TP53. It tests the dominant negative effect of mutant TP53 versus that of the endogenous TP53. The wildtype p53 protein in the cells of the first score is less affected by truncate p53 protein or p53 protein with driver in the tetramerization domain. This reduction occurs because wildtype p53 proteins do not create non-functional tetramers with the mutant p53, thus leading to only wildtype p53 tetramers and results in false negative values in the first score. Indeed, the TVA correlation with the Giacomelli's first score only for missense variants in the DNA binding domain was much stronger (ρ=0.72>0.53) and TVA ranked first compared to all 31 predictors (FIG. 5). Variants' scores distribution varies among DMS studies. Some are more polarized while others have a wider distribution of values. As data is more polarized into maximal and minimal values it reinforces the dichotomous approach of drivers and passengers, while a wide distribution of values is more suitable for the spectral effect approach. In TP53, for example, Kotler's score (FIG. 7) is more polarized, while Kato's (FIG. 23) and Giacomelli's scores (FIGS. 6, 24, 25) are more spectral. For the spectral scores the distribution contains one extreme of neutral variants with normal protein function, one extreme of pathogenic variants with abnormal protein function and many intermediate variants. Good correlations were found of TVA and the gap of intermediate variants between the two extremes of DMS scores distribution. This suggests that the relative intermediate prevalence of these variants might be explained by partial protein function caused by weak\moderate drivers while the two extremes represent functional and non-functional protein variants relating with passengers and strong drivers respectively. These weak to moderate drivers are part of the long tail of drivers that the approach can discover (FIG. 22). Some deviations can be found in each MDS assay score and TVA graph (further information and analysis can be found in the other figures).

### Example 4: Overall Survival in TVA Subgroups in Prognostic Genes

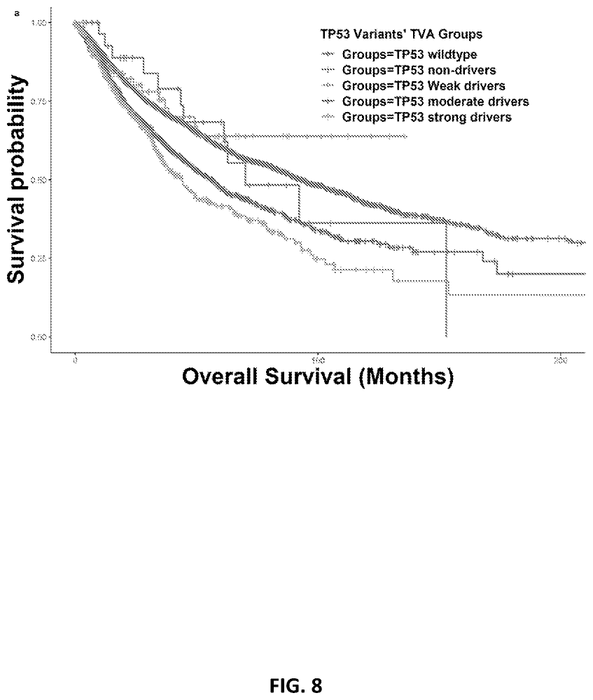

The appearance of variants in certain cancer genes can serve as a prognostic indicator. One such gene is TP53 gene, which is associated with poor prognosis in a variety of cancer types. Tumors with more than one variant were excluded to avoid ambiguity. All TCGA samples with one unique TP53 variant were divided into four groups: non-drivers, weak, moderate, and strong drivers according to their TVA values and binomial test catalogue label (see Materials and Methods). These groups were compared with a control group of patients with wildtype TP53 (Table 6, FIG. 8). The analysis showed distinct overall survival (OS) curves for each TP53 variants group which was well correlated with the variants strength as estimated by TVA. Non-drivers and weak drivers had the best OS from all TP53 groups. Non-drivers had no significant difference in comparison to other groups due to small sample size (n=32), but patients with weak drivers had statistically significant better survival rates compared to patients with moderate and strong drivers (p-value=0.03 and 0.005, respectively, log rank test) although having a small sample size (n=77). Both non-drivers and weak drivers were comparable to the OS curve of patients with wildtype TP53 (p-value=0.88 and 0.46, respectively, log rank test). Patients with moderate drivers had worse OS compared to weak drivers (p-value=0.02, log rank test), wildtype (p-value=1.6e-14, log rank test) and non-drivers (p-value=0.37, log rank test). Patients with strong drivers had the worst OS of all groups (p-value=3.5e-14 and 0.0047 for wildtype and weak drivers respectively, log rank test), with marginal significance comparing to moderate drivers (p-value=0.07, log rank test).

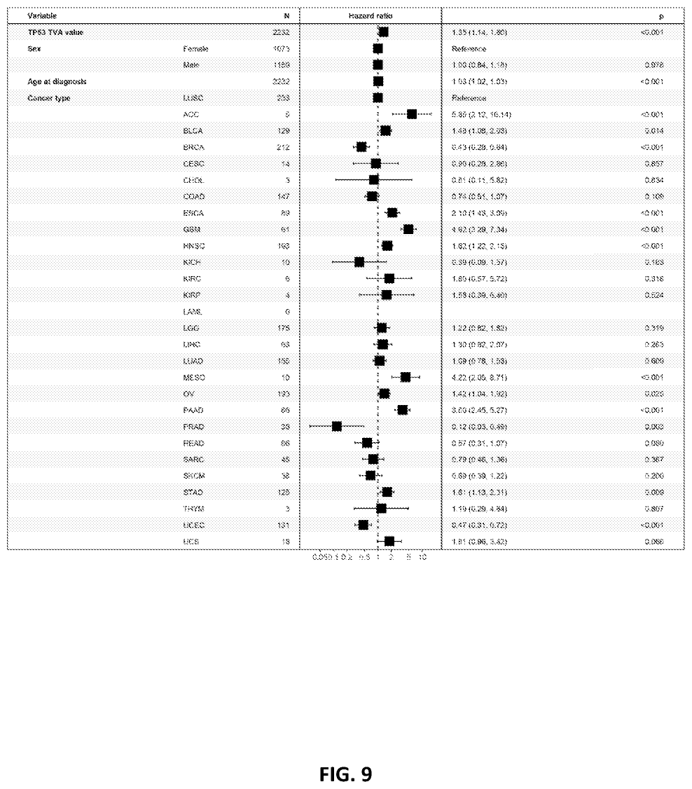

The association between the continuous value of TVA and OS for patients with any single TP53 variant was investigated. A multivariable analysis was performed that included: age of diagnosis, sex, and cancer type. A strong effect of the TVA value on OS was found where higher TVA was associated with shorter OS, from the other variables tested (HR=1.35, p-value=0.000478) (FIG. 9). Note that FIG. 9 uses the hazard ratio rather than the odds ratio.

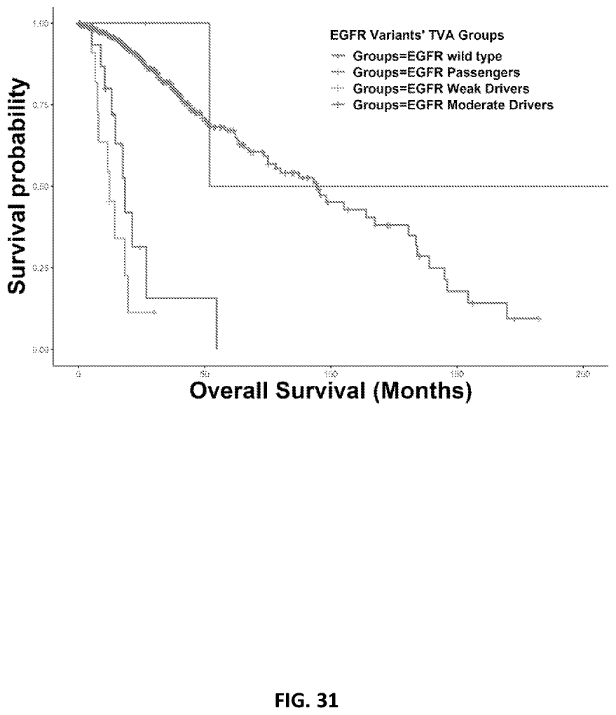

It is expected that similar trends will be obtained with other genes with prognosis implication in specific cancers, but the sample size of TCGA data is insufficient in most cases due to the tumor type specificity of the gene or due to the low frequency of mutations. For example, Lower Grade Glioma (LGG) patients with EGFR have poor OS. In the LGG survival analysis all groups have very small sample size (non-drivers n=3, weak n=11, moderate n=16, strong n=0), non-drivers tended to have a good survival curve, comparable with wildtype EGFR group (p-value=0.324, log rank test), and with distinction of a worse prognosis of weak drivers (p-value=0.04, log rank test), and moderate drivers (p-value=0.08, log rank test). Both weak and moderate drivers were distinct compared to the wildtype EGFR group (p-values<1e-13, log rank test), with no clear distinction between the individual drivers' groups (p-value=0.198, log rank test) (Table 2, FIG. 31).

TABLE 6
Pairwise comparison using log rank test

| | | | | TP53 |
|---|---|---|---|---|
| | TP53 | TP53 non- | TP53 Weak | Moderate |
| | wildtype | drivers | Drivers | Drivers |
| | (n = 7,790) | (n = 32) | (n = 77) | (n = 1,650) |
| TP53 | 0.8825 | — | — | — |
| non-drivers | | | | |
| (n = 32) | | | | |
| TP53 Weak | 0.4598 | 0.7993 | — | — |
| Drivers | | | | |
| (n = 77) | | | | |
| TP53 | 1.6e−14 **** | 0.3668 | 0.0297 * | — |
| Moderate | | | | |
| Drivers | | | | |
| (n = 1,650) | | | | |
| TP53 Strong | 3.5e−14 **** | 0.1335 | 0.0047 ** | 0.0696 + |
| Drivers | | | | |
| (n = 574) | | | | |

~0 = ‘****’,
<0.001 = ‘***’,
<0.01 = ‘**’,
<0.05 = ‘*’,
<0.1 = ‘+’

### Example 5: Drug Sensitivity by TVA

Rare pathogenic variants are becoming important in the inter-individual variability in drug response. Identification of those variants and interpretation of their pathogenicity is essential for pharmacogenetic predictions. The Genomics of Drug Sensitivity in Cancer Project (GDSC) is a public database including information on the response of numerous human cancer cell lines to a wide range of anti-cancer drugs. In an analysis, the recently published GDSC2 dataset was used which is considered as an improved and more accurate source compared to the previous edition. GDSC2 includes 809 cell lines and 198 compounds tested with 135,242 IC50 calculations. Genomic features and drug response associations were analyzed from GDCS's analysis of variance model that met certain criteria (see Materials and Methods). In order to further inspect each variant's TVA association with drug response all variants were divided into sub-groups: (i) non-drivers (ii) weak drivers (iii) moderate drivers (iv) strong drivers (v) very strong drivers and (vi) wildtype (see Materials and Methods). Part of the drugs tested in GDSC2 directly affect the protein translated from the associated cancer gene alterations while others affect indirectly through the cancer gene pathway (upstream or downstream to the gene).

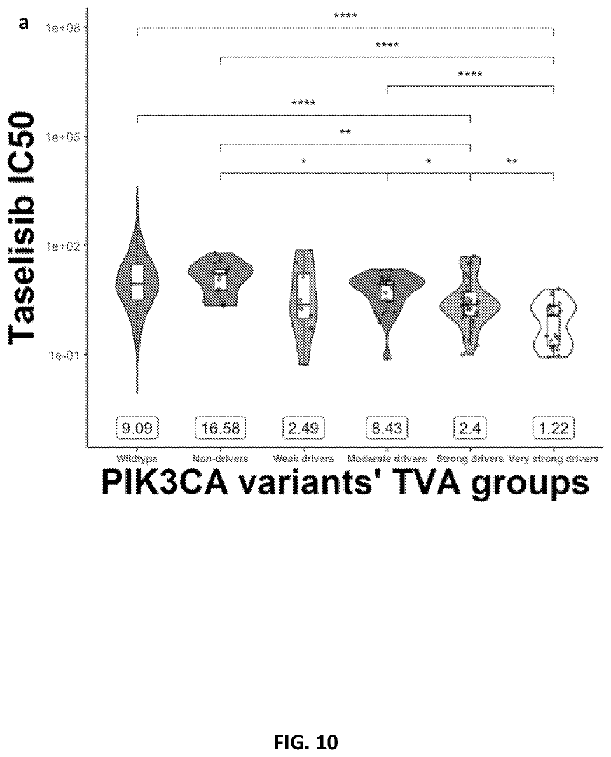

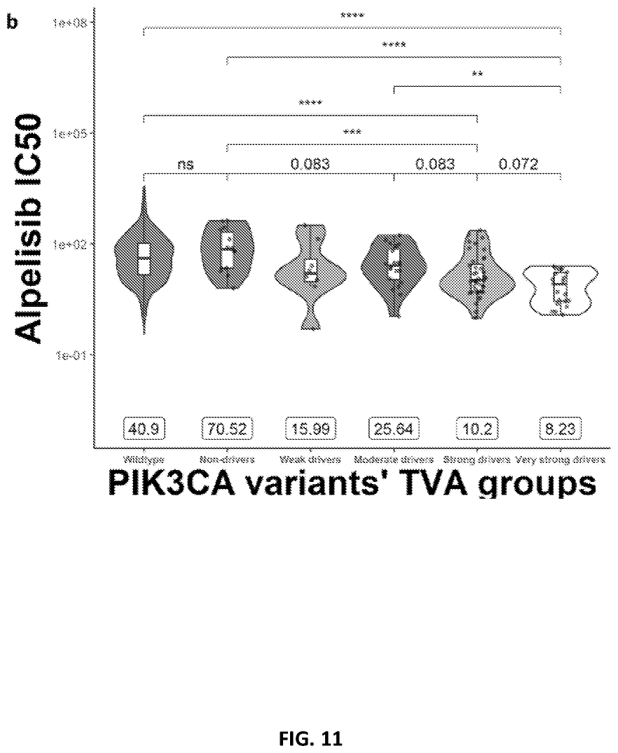

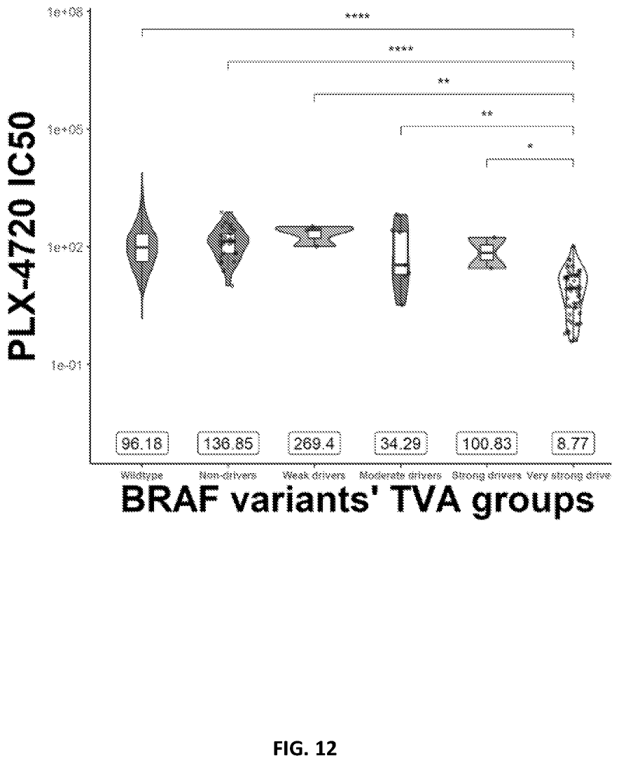

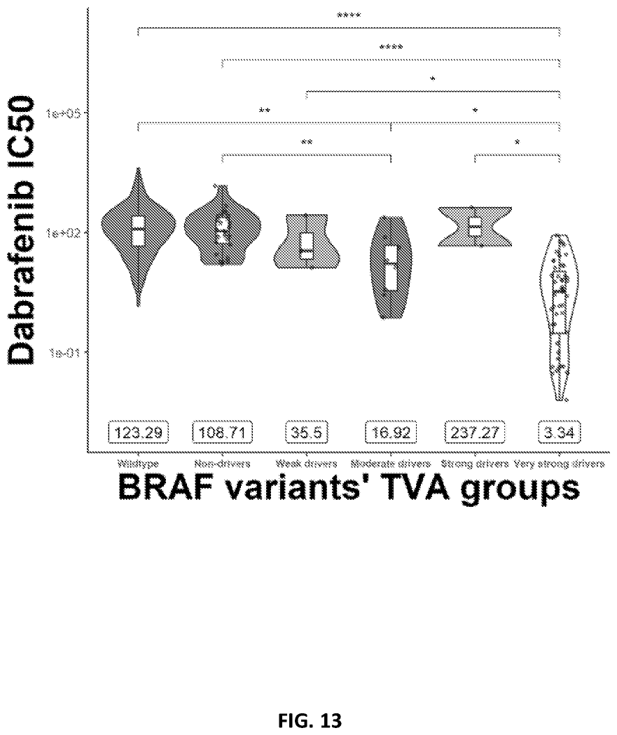

PIK3CA gene encodes the catalytic subunit of PI3K. A strong association was found between TVA's sub-groups of PIK3CA variants and response to two different PI3K inhibitors (FIGS. 10, 11). On the other hand, TVA's sub-groups of BRAF variants had different association with various BRAF inhibitors. For PLX4720 inhibitor (Vemurafenib precursor compound) only the “Very Strong Drivers” group had distinct low IC50 while all other groups were all comparable to each other (FIG. 12). “Very Strong Drivers” group includes V600E class I variant and all other drivers group include both class II and III BRAF variants. It is known that this inhibitor works only on class I, RAS-independent monomers, and not on class II and III variants. For Dabrafenib, another BRAF inhibitor, TVA's sub-groups of BRAF variants were associated with drug response, except for two cell lines in “Strong Drivers” group (FIG. 13). Indeed, there are indications that Dabrafenib has a partial response to tumors with BRAF non-class I variants.

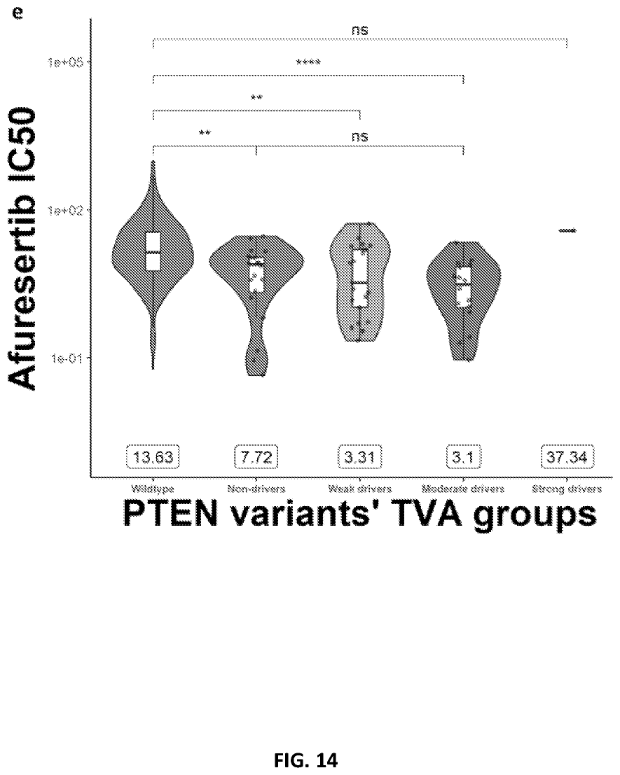

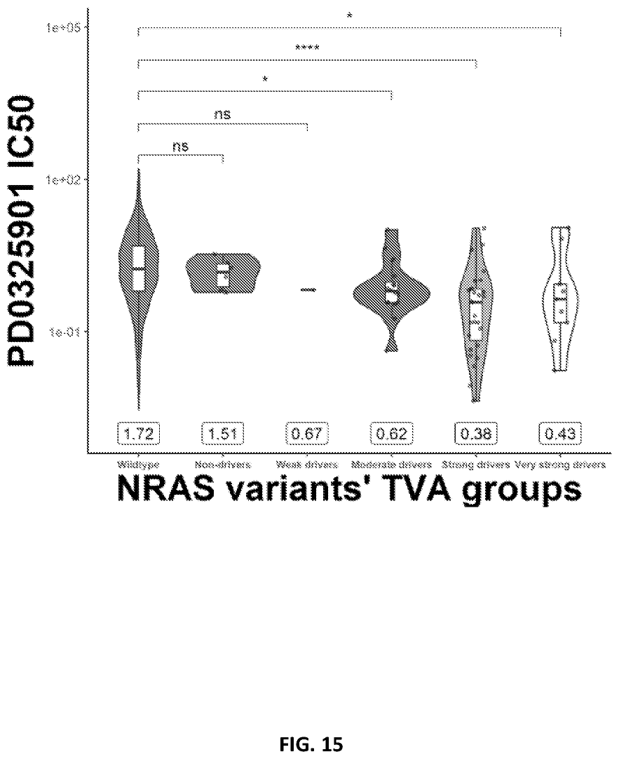

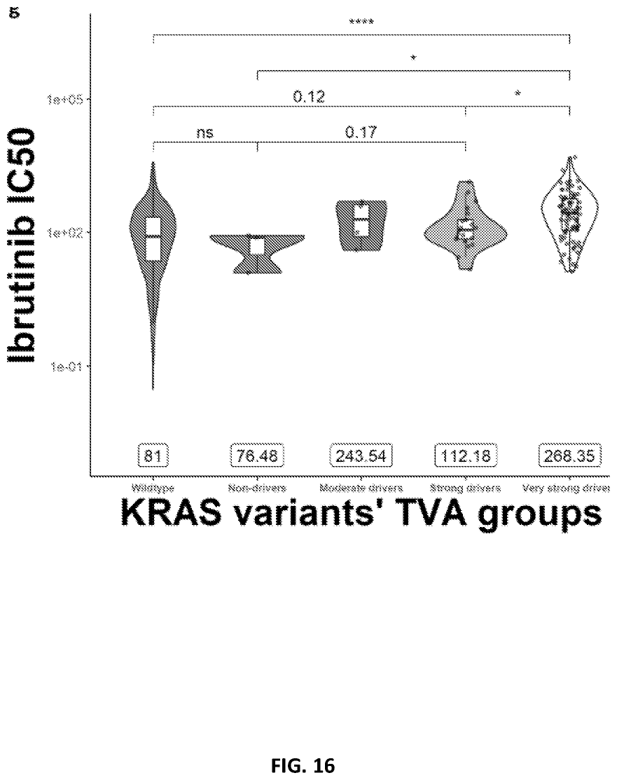

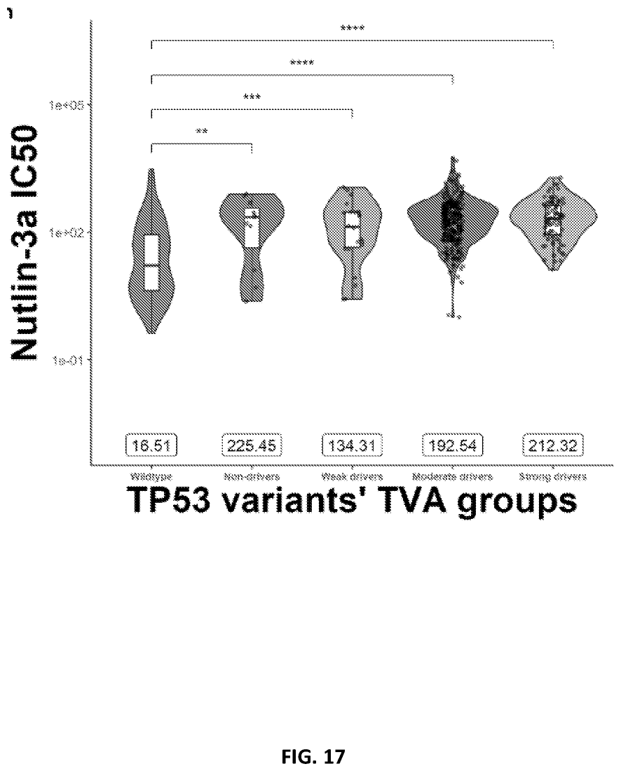

As for indirect inhibitors which affect downstream to the gene, association to variants' pathogenicity could be related to (i) the number of genes between the mutated gene and the drug target gene in the pathway, (ii) dispersion of the effect of the mutated gene into many pathways. PTEN is the main negative regulator of the PI3K-AKT pathway, therefore it is reasonable that variants' pathogenicity would have association with AKT inhibitors. A weak association was identified between TVA's sub-groups of PTEN and AKT inhibitor, except for one outlier cell line with R130G, a well-known driver variant in the “Strong Drivers” group (FIG. 14). On the other hand, TVA's sub-groups of NRAS variants in association with MEK inhibitor had a distinction only between drivers and non-drivers with no differences between all drivers' sub-groups (FIG. 15). NRAS has three main downstream effector pathways of which RAF-MEK-ERK is only one. This dispersion and genes distance in pathway could be the reason for lower association to NRAS variants' pathogenicity. For indirect inhibitors upstream to the gene, a worse response can be expected for stronger drivers of the gene. Indeed, a weak association was identified between TVA's sub-groups of KRAS and BTK inhibitor (FIG. 16). On the other hand, TVA's sub-groups of TP53 variants association with MDM2 inhibitor was only between any TP53 variant and wildtype TP53 with no distinct differences between all TP53 variants' sub-groups (FIG. 17).

### Example 6: POLE Variants' TVA Values Correlation to Tumor Variants Count

The POLE gene encodes the catalytic subunit of DNA polymerase F, which is involved in DNA repair and chromosomal DNA replication. Driver variants in DNA polymerase F result in hyper-mutant cancers. Different driver variants of POLE's induce different mutation signatures. The three most frequent pathogenic variants are P286R, V411L and S459F, each related to a different POLE signature—SBS10a, SBS10b and SBS28 respectively. The tumor mutation burden (TMB) for some samples with POLE variants is low and comparable to tumors without a POLE variant, while for other POLE variants the TMB is high. This indicates that some POLE variants might be passengers. A recent study investigated all POLE variants in TCGA endometrial carcinoma samples and mapped the pathogenic variants. Indeed, the catalogue contains almost all predicted pathogenic variants (10/11) in this disclosure, and the missing variant is marginally above the threshold of the adjusted p-value (0.11).

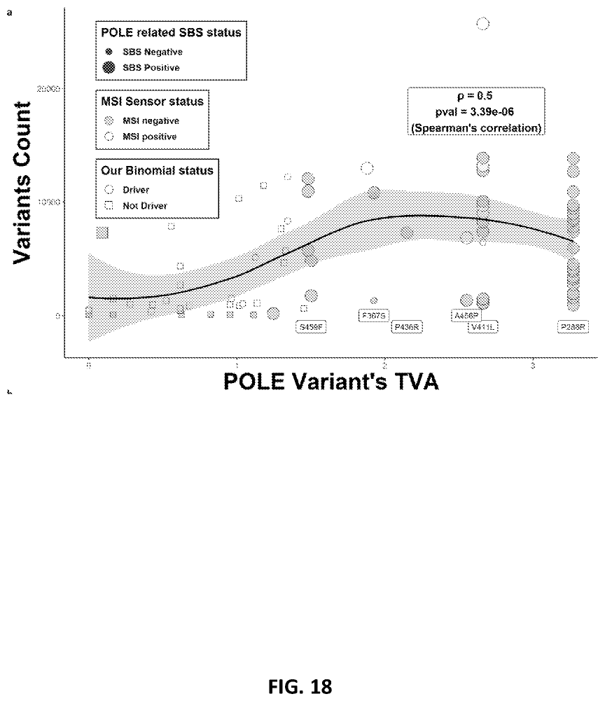

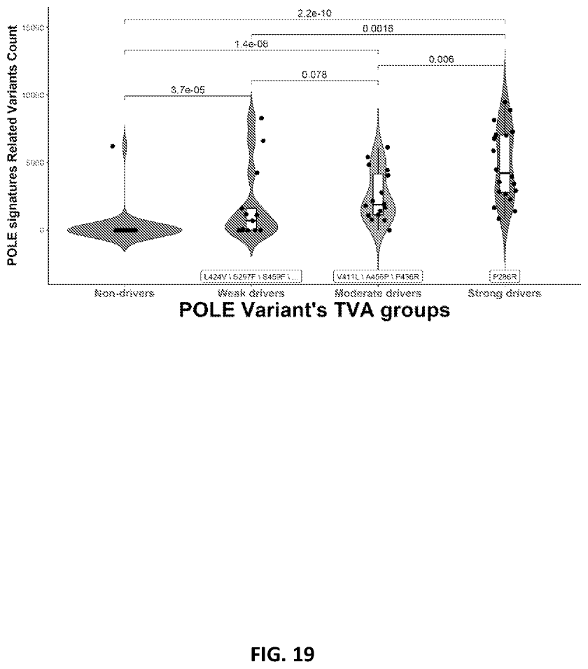

The POLE variants are usually dichotomized as pathogenic or non-pathogenic, and only a few studies investigated the effect size of each pathogenic variant on the total TMB. The correlations were examined between TMB and the POLE variants in TCGA endometrial carcinoma (since several POLE variants may co-exist in a single sample, for these cases the POLE variant with the highest TVA value was selected). The analysis (FIG. 18) shows positive correlation (ρ=0.5, p=3.39e-06, Spearman's correlation) between samples TMB and POLE variant TVA value. Most samples with high TMB and POLE variants with low TVA have microsatellite instability (MSI) according to high “MSI sensor score”. MSI by itself causes a large number of variants due to DNA mismatch repair deficiency, which accounts to the high variants count in samples with low POLE TVA value. Co-existence of POLE known driver variants and microsatellite instability is relatively rare, and this appears to be true for samples with a high POLE TVA value in the analysis. POLE related signatures were also further enriched in samples with POLE driver variants. The effect was tested of different POLE drivers on counts of POLE related variants only. For this analysis samples were grouped according to TVA into POLE non-drivers, weak drivers, moderate drivers, and strong drivers (see Materials and Methods). For each tumor the count of POLE related variants according to POLE's signatures was summarized (see Materials and Methods). This analysis confirmed a distinction between different TVA groups with statistical significance (FIG. 19). By using only variants from POLE related signatures, a clearer look at the genuine effect of each driver was obtained, without masking other reasons for large variants count such as MSI and MMR. Same correlation between variants frequency and mutational rate was reported in a recent yeast assay but only for variants in POLE's DNA binding cleft.

### Example 7: Analysis of Variant Mutational Rates

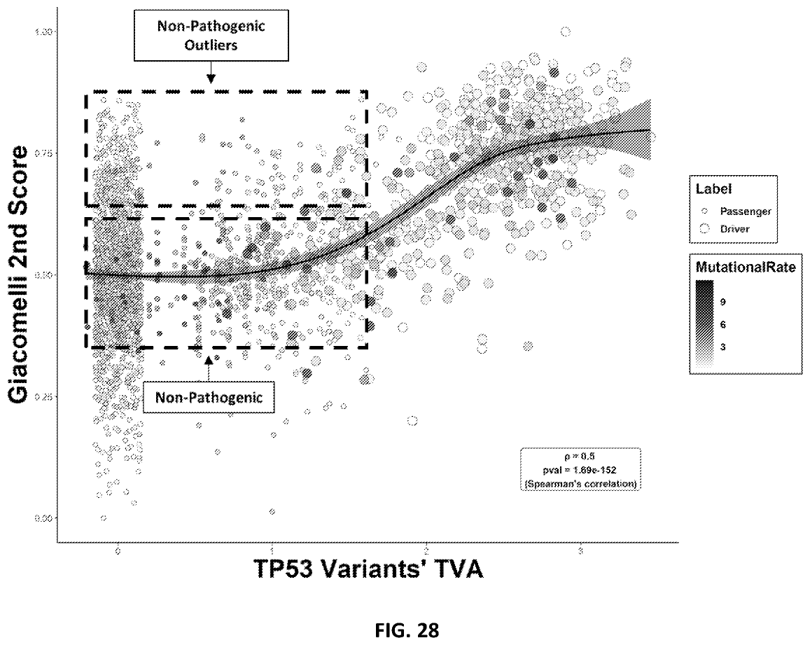

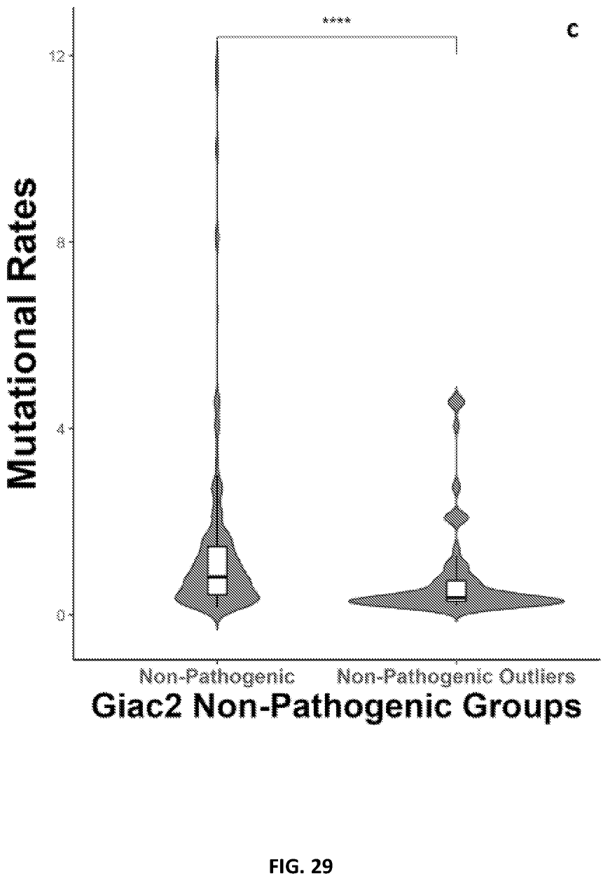

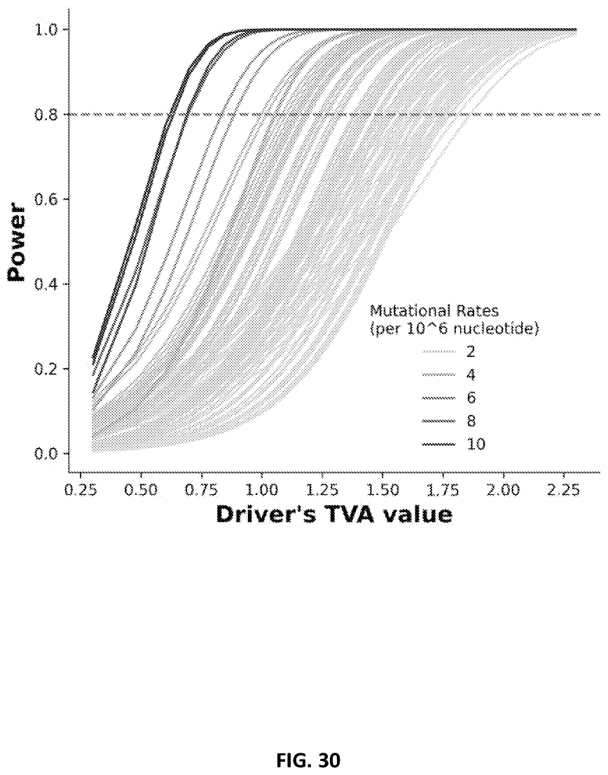

In every study there are variants deviating from the correlation. This deviation can be explained in most cases by assay's experimental methodological limitations or TVA statistical limitations. One group of exceptions are variants with high TVA values and normal functional scores. An example of a methodological limitation is seen in TP53 E294X, a known nonsense driver with high TVA value (2.97) but Giacomelli's first score predicts it as normal activity (0.4) due to methodological limitations as presented above. An example of a statistical limitation is seen in TP53 D391A, a variant predicted as normal activity in all experimental scores but with a moderate TVA value (1.6). This is caused due to a very low mutational rate, and as expected the variant is not statistically significant in the adjusted binomial test (raw p-value=0.024, adjusted p-value=1.0). Other group of exceptions are variants with low TVA values but loss of function experimental scores. An example of a methodological limitation is seen in TP53 I232L, a variant with imputed TVA value of 0 and predicted as having a normal function in Giacomelli and Kotler scores but predicted as having loss of function score in Kato's score. This disagreement could result from Kato's yeast model as compared to human cell tissue on all other assays. An example of a statistical limitation is many variants with very low TVA, some with loss of function scores and some with normal function as measured by Giacomelli's second score (FIG. 28). This can be caused by variants in positions with low mutational rate. For the low TVA variant group, the mutational rates in the loss of function group were found to be significantly lower than in the normal function group (p-value=2.9e-9, t test) (FIG. 29). Accordingly, variants in low mutational rates positions simply do not have enough power to reach statistical significance for weak drivers and this might be the cause for the discrepancy (FIG. 30).

### Example 8: HRAS and CS

Costello syndrome (CS) is a rare genetic disorder caused by mutations in the HRAS gene. This disorder is characterized by distinctive facial features, short stature, and an increased risk of certain types of cancer (PMID: 16170316). The TVA distribution of all known germline RASopathies variants labeled by HGMD was analyzed. Most of these variants are CS. These variants were compared to those identified as somatic in the CSD and the variants were divided into drivers with binomial test adjusted p-values below 0.1 and variants which were not significant.

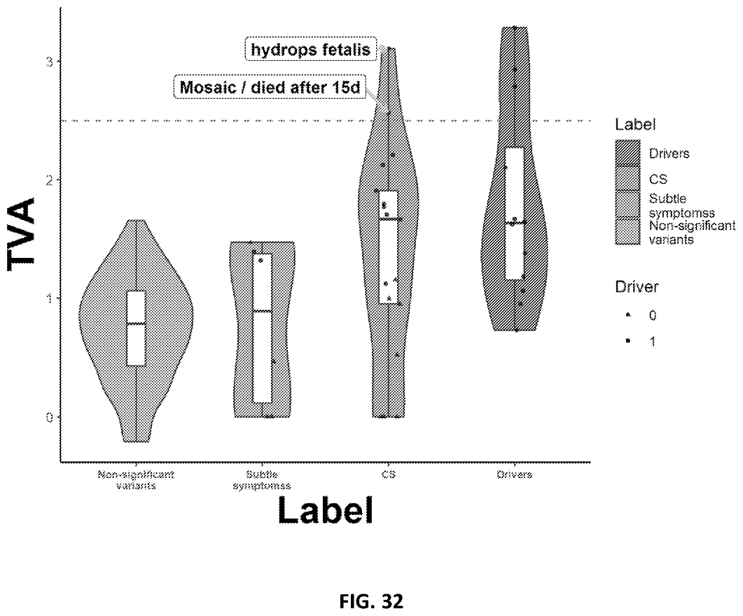

As expected, TVA values was correlated to the groups. The drivers group which is not labeled in HGMD had the highest TVA values. The second highest value was for the CS group and third highest, the group of more subtle RASopathies syndromes (FIG. 32). Variants with TVA values above 2.5 are well known hotspot drivers in cancer but are rarely seen in patients with RASopathies. Two of the CS variants with such TVA levels are not classical CS variants. The first variant was found in a dead embryo with hydrops fetalis (PMID: 33027564); the second one was found in two cases—a fetus with hydrops that died after 15 days (PMID: 32732226), and a mosaic patient who was not seriously affected by this strong variant (PMID: 34109654). It is well known that mosaic RASopathies cause more defined defects which are restricted to specific tissues (PMID: 30007125). By contrast, most CS variants have TVA values ranging between 1 and 2, and many were classified as drivers by the binomial test.

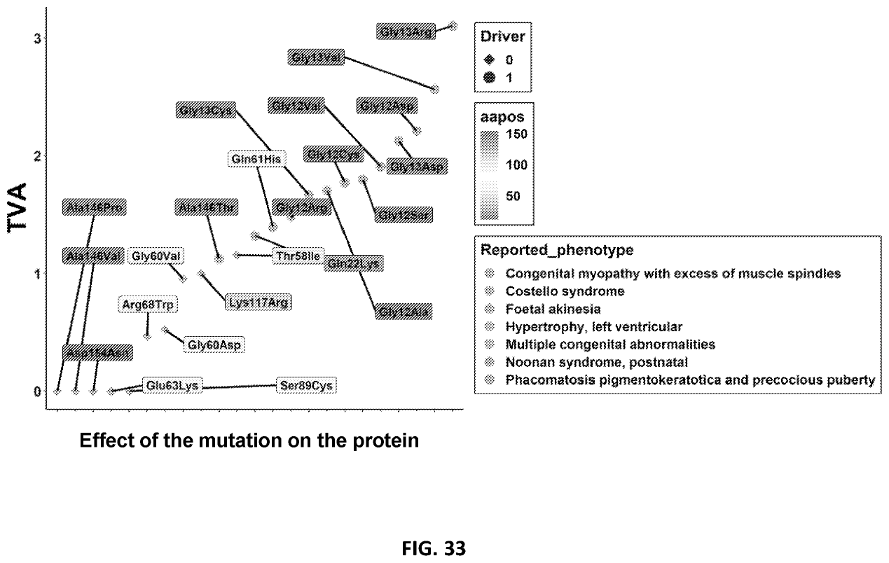

CS is typically associated with amino acid variants in position 12/13, while variants in other amino acid positions exhibit less obvious symptoms (PMID: 28328122). Sub-group analysis of HGMD variants based on TVA found that variants in positions 12/13 have a higher TVA value than those in other positions (FIG. 33). A higher TVA reflects a higher selection for cancer, which is coupled with a stronger effect on protein. Therefore, position 12/13 CS patients display more classic symptoms while weaker variants display more mild symptoms.

Hence, the TVA values can stratify the risk to develop cancer among different mutations associated with CS. This identification will contribute to personalized follow-up of the patients

### Summary/Discussion

In an example, a catalogue of 10,866 driver variants was created from 535 cancer genes based on a binomial test adjusted p-value and a new measure called TVA was calculated representing the selective power for each variant. These findings show that TVA is highly correlated with the biological activity strength of driver variants in many different laboratory and clinical validations. TVA was highly correlated with functional scores of five different DMS experiments measuring the effect of different variants in TP53 and PTEN genes. It also outperformed 31 computational predictors in most studies. This high correlation suggests that TVA represents cancer pathogenicity better than other computational scores, and thus can be used as a measure of driver variants' pathogenicity and biological activity strength for cancer variants. In pharmacological data, TVA was correlated with drug sensitivity in several cancer genes that are either directly or indirectly affected by these drugs. Hence, TVA may contribute to predict drug response for non-classic driver variants. Positive correlation of TVA was also shown in two clinical examples: (i) for POLE gene, TVA had positive correlation with POLE related (according to genomic context signatures) tumor variants count; (ii) for TP53 gene, TVA had positive correlation to overall survival both in TVA's sub-groups and as a continuous parameter.

This disclosure is novel in both the amount of driver variants identified, and in the quantitative measure of cancer variants effect with TVA. This was extensively validated by data from many different sources, representing the strength and credibility of TVA. the findings reinforce the paradigm that variant pathogenicity is much more complex than the dichotomic classification to drivers and passengers and that variants' effect on quantification methods can be useful for clinical purposes. All the validations demonstrated that TVA can be used for comparison of variants in the same gene. TVA can also be used for comparison of variants of different genes as it measures variants' selective power in the same manner for all cancer genes as opposed to methods based on many different DMS data for each gene. Thus, TVA might be well suited for pathogenicity prediction regardless of gene specific mechanisms since positive selection can result from many different mechanisms.

Several conceptual approaches have been used to quantify variants' effects. Some try to estimate properties of specific mechanisms such as protein stability while others predict the combined effect of many mechanisms. The mechanistic specific approaches are useful to distinguish and explain drivers' pathogenicity mechanisms, but by doing so they limit the predictive power of other mechanisms. A more general approach has the advantages of capturing many biologically relevant effects of variants, to potentially increase the accuracy of pathogenicity prediction. Several studies presented different implementations of general approaches: DMS experiments on selected proteins, supervised machine learning on DMS data with biochemical, structural, and sequence-based features, unsupervised machine learning based on context-dependent constraints in biological sequences, and selection intensity based on cancer cell lineages. Every approach encompasses its own limitations: (i) DMS studies are expensive, time consuming and limited to a specific gene for every study; (ii) Supervised machine learning approaches such as Envision are trained on a small number of selected DMS studies that were comprehensive enough and need to normalize scores from many genes with different variant effect measures and protein properties. Comparisons to other predictors found that the Envision tool produced moderate overall correlation performance for human DMS data although it was trained for that purpose; (iii) Unsupervised tools based on context-dependent constraints such as EVE and DeepSequence lack information on many proteins' positions and nonsense mutations due to methodological reasons. It may be misleading for variants affecting RNA such as splicing variants, and in some genes does not perform well as shown in EVE's paper; (iv) The disclosure based on “selection intensity” of somatic variants in cancer cell lineages included only a small number of cancer samples in their calculations, separated the predictions by cancer type although it was usually unnecessary, focused primarily on known strong drivers, and did not validate their findings in any clinical setting. In the current disclosure, a large number of sequenced cancer samples were relied on, including information from healthy population databases, using a binomial test threshold for statistical significance of pathogenic variants, predicting variants' effects for 535 cancer genes, and validating the variants' quantitative effect by numerous laboratory and clinical scopes. In addition, the TVA measure is not dependent on a particular mechanism, leading to both higher accuracies also for variants affecting RNA. For example, TP53 E224D has TVA value of 2.1 and is known as deleterious for TP53 splicing.

Tumors usually harbor many variants, and it is important to determine which are drivers, and which are more important for tumor survival. As more therapies are being developed to target more cancer genes, it is important not only to recognize the pathogenic variants but also to prioritize which variants are more important to the tumor survival. The catalogue and TVA can be used to both recognize driver variants and to prioritize them according to their selective variant effect. This prioritization might contribute for prognosis as well as for the selection of adequate combination therapies for the tumor's more important driver variants. This method might be especially suitable for the assessment of different genes variants as all calculations are based on selection power.

While the foregoing written description of the invention enables one of ordinary skill to make and use what is considered presently to be the best mode thereof, those of ordinary skill will understand and appreciate the existence of alternatives, adaptations, variations, combinations, and equivalents of the specific embodiment, method, and examples herein. Those skilled in the art will appreciate that the within disclosures are exemplary only and that various modifications may be made within the scope of the present invention. In addition, while a particular feature of the teachings may have been disclosed with respect to only one of several implementations, such feature may be combined with one or more other features of the other implementations as may be desired and advantageous for any given or particular function. Furthermore, to the extent that the terms “including”, “includes”, “having”, “has”, “with”, or variants thereof are used in either the detailed description and the claims, such terms are intended to be inclusive in a manner similar to the term “comprising.”

Other embodiments of the teachings will be apparent to those skilled in the art from consideration of the specification and practice of the teachings disclosed herein. The invention should therefore not be limited by the described embodiment, method, and examples, but by all embodiments and methods within the scope and spirit of the invention. Accordingly, the present invention is not limited to the specific embodiments as illustrated herein, but is only limited by the following claims.

What is claimed is:

1. A method for quantitatively assessing a biological effect of at least one gene variant of a subject using a computer system comprising a processor, memory, and instructions stored in the memory, which, when executed by the processor, perform the method comprising:
receiving the at least one gene variant of the subject;
analyzing a genomic database to determine a mutation rate for the at least one gene variant;
determining an observed number of occurrences of the at least one gene variant in the database;
calculating an expected number of occurrences of the at least one gene variant based on the mutation rate and the observed number of occurrences;
calculating a predictor associated with the at least one gene variant based on the mutation rate, the observed number of occurrences and the expected number of occurrences;
using the predictor to generate a quantitative assessment of the biological effect of the at least one gene variant; and
transmitting the predictor and the quantitative assessment to a user device.

2. The method of claim 1, wherein the quantitative assessment comprises a prognosis, a risk of developing cancer, or a treatment response.

3. The method of claim 1, wherein the predictor comprises a tumor variant amplitude (TVA), said TVA being equal to a logarithm of a ratio of the observed number of occurrences of the at least one gene variant in the genomic database divided by the expected number of occurrences of the at least one gene variant in the genomic database.

4. The method of claim 1, wherein, prior to analyzing the genomic database, the genomic database is filtered to avoid duplication of samples from the same subject and also filtered using at least one of:
a genomic coordinate of each entry;
a nucleotide alteration of each entry;
a somatic status of each entry; or
a type of cancer of each entry.

5. The method of claim 1, wherein the quantitative assessment comprising the steps of:
comparing a plurality of drug therapies of tumors with gene variants present in the tumors;
identifying, based on the comparison, a selected drug therapy of the plurality of drug therapies for use with a subject's tumor; and
predicting, based on the comparison, the likely response of the subject's tumor to the selected drug therapy.

6. The method of claim 5, wherein identifying the selected drug therapy of the plurality of drug therapies comprises prioritizing gene variants based on a classification of the gene variants and based on the TVA.

7. The method of claim 1, wherein the quantitative assessment comprises the steps of:
comparing a subject's germline DNA with a database of gene variants and cancer risk; and
quantifying, based on the comparison, a risk that a subject will develop a cancer.

8. The method of claim 1, wherein the quantitative assessment comprises the steps of:
comparing a subject's tumor DNA with a database of gene variants and tumor mutations; and
quantifying, based on the comparison, a prognosis for a subject.

9. The method of claim 1, further comprising using the predictor as an input to an artificial intelligence model for determining a diagnosis.

10. A system for quantitatively assessing a biological effect of at least one gene variant of a subject, for use with a user device, comprising:
a measurement device;
a processor; and
memory accessible by the processor and storing computer program instructions which, when executed by the processor, perform a method of:
measuring, by the measurement device, a number of occurrences of the at least one gene variant;
analyzing, at the processor, a genomic database to determine a mutation rate for the at least one gene variant;
determining, at the processor, an observed number of occurrences of the at least one gene variant in the database;
calculating, at the processor, an expected number of occurrences of the at least one gene variant based on the mutation rate and the observed number of occurrences;
calculating, at the processor, a predictor associated with the at least one gene variant based on the mutation rate, the observed number of occurrences and the expected number of occurrences;
using the predictor, at the processor, to generate a quantitative assessment of the biological effect of the at least one gene variant; and
transmitting the predictor and the quantitative assessment to the user device.

11. The system of claim 10, wherein the quantitative assessment comprises a prognosis, a risk of developing cancer, or a treatment response.

12. The system of claim 10, wherein the predictor comprises a tumor variant amplitude (TVA), said TVA being equal to a logarithm of a ratio of the observed number of occurrences of the at least one gene variant in the genomic database divided by the expected number of occurrences of the at least one gene variant in the genomic database.

13. The system of claim 10, wherein the processor, prior to analyzing the genomic database, filters the genomic database to avoid duplication of samples from the same subject and also filters the genomic database using at least one of:
a genomic coordinate of each entry;
a nucleotide alteration of each entry;
a somatic status of each entry; or
a type of cancer of each entry.

14. The system of claim 10, wherein the quantitative assessment comprises the steps of:
comparing a plurality of drug therapies of tumors with gene variants present in the tumors;
identifying, based on the comparison, a selected drug therapy of the plurality of drug therapies for use with a subject's tumor; and
predicting, based on the comparison, the likely response of the subject's tumor to the selected drug therapy.

15. The system of claim 14, wherein the processor identifies the selected drug therapy of the plurality of drug therapies by prioritizing gene variants based on a classification of the gene variant and based on the TVA.

16. The system of claim 10, wherein the quantitative assessment comprises the steps of:
comparing a subject's germline DNA with a database of gene variants and cancer risk; and
quantifying, based on the comparison, a risk that a subject will develop a cancer.

17. The system of claim 10, wherein the quantitative assessment comprises the steps of:
comparing a subject's tumor DNA with a database of gene variants and tumor mutations; and
quantifying, based on the comparison, a prognosis for a subject.

18. The system of claim 10, wherein the processor further uses the predictor and an artificial intelligence model to determine a diagnosis.
